(19) United States
(12) Patent Application Publication
(10) Pub. No.: US 2023/0399295 A1
(43) Pub. Date: Dec. 14, 2023

(54) COMPOSITIONS FOR INCREASING TISSUE REGENERATION AND DELAYING OR REDUCING GRANULOMA FORMATION

(71) Applicant: THE BRIGHAM AND WOMEN'S HOSPITAL, INC., Boston, MA (US)

(72) Inventors: Ashley E. SHAY, State College, PA (US); Robert NSHIMIYIMANA, Boston, MA (US); Charles N. SERHAN, Needham, MA (US)

(73) Assignee: THE BRIGHAM AND WOMEN'S HOSPITAL, INC., Boston, MA (US)

(21) Appl. No.: 18/035,337

(22) PCT Filed: Nov. 4, 2021
(86) PCT No.: PCT/US2021/058073

§ 371 Date: May 4, 2023

Related U.S. Application Data
(60) Provisional application No. 63/109,370, filed on Nov. 4, 2020.

(51) Int. Cl. C07C 323/59; A61K 47/64; C07C 237/52; A61P 27/02

(52) U.S. Cl. CPC C07C 323/59; A61K 47/64; C07C 237/52; A61P 27/02

(57) ABSTRACT

Disclosed are compounds and methods to enhance the resolution of inflammation and inflammatory diseases comprising novel compounds derived from conjugated specialized pro-resolving lipid mediators (SPMs). These compounds are useful in preventing and treating granuloma formation and subsequent loss of tissue regeneration and organ function. The compounds are 5 amino conjugates of Resolvin D3 and Resolvin D4 with glutathione. Further identified are novel glutathione-conjugated specialized pro-resolving lipid mediators. These newly identified cysteinyl-SPM significantly increased the amount of tissue regenerated (p<0.05) and accelerated the speed of regeneration by ˜24 h in Planarian regeneration. Together, these results provide evidence for a 4(5) epoxide resolvin pathway in the biosynthesis of novel 4,5 cysteinyl-SPMs that reduce granuloma and are useful for treating or preventing diseases including Mycobacterium tuberculosis, chronic granulomatous disease, granulomatous steatitis, foreign-body granulomas, interstitial lung fibrosis (ipf), leprosy, arthritis, sarcoidosis, liver fibrosis, heart fibrosis, renal fibrosis, hepatic cirrhosis, pulmonary fibrosis and organ fibrosis.

### CROSS REFERENCE TO RELATED APPLICATIONS

This application claims priority to U.S. Provisional Application No. 63/109,370, filed Nov. 4, 2020, which is hereby incorporated by reference herein.

### STATEMENT REGARDING FEDERALLY SPONSORED RESEARCH

The work leading to this invention was supported, in part, by National Institutes of Health (NIH) grant P01GM095467. The United States Government may have certain rights in this invention.

### FIELD OF THE INVENTION

This invention relates to compounds, compositions, and methods of use for significantly increasing tissue regeneration, and delaying or reducing the advancement of granuloma formation.

### SUMMARY OF THE INVENTION

This invention provides compounds, compositions, and methods of use for accelerating the speed of tissue regeneration, and for delaying or reducing the advancement of granuloma formation.

The acute inflammatory response is essential for host defense and requires active resolution to return to homeostasis. Failed resolution plays a major role in the development of chronic inflammation and the etiology of multiple diseases such as for example arthritis, periodontitis, thrombosis, fibrosis, and cancer. The resolution of inflammation is an active process driven by specialized pro-resolving lipid mediators (SPM) that arise from polyunsaturated fatty acids such as arachidonic acid (AA), docosahexaenoic acid (DHA), eicosapentaenoic acid (EPA), and docosapentaenoic acid (DPA).

SPMs counter-regulate pro-inflammatory mediators, increase uptake of bacteria, cellular debris, and apoptotic neutrophils, and decrease the influx of infiltrating leukocytes. Granuloma formation occurs during inflammation when the immune system is unable to eliminate bacteria, fungi, or a foreign body and instead forms an enclosure.

Chronic granuloma formation leads to necrotic lesions, loss of tissue regeneration, loss of tissue/organ function, and potentially death. The role of specialized pro-resolving lipid mediators in granuloma formation and subsequent loss of tissue regeneration and organ function is not known. Here we identified a novel peptido-lipid pro-resolving mediator, 4,5-Resolvin Conjugate in Tissue Regeneration-1 (4,5-RCTR-1), that significantly delays granuloma formation as well as increases the amount of tissue regenerated and accelerates the time required for tissue regeneration.

We disclose herein that human neutrophils (PMNs), human macrophages (M 1), and human recombinant enzymes, biosynthesize a novel peptido-lipid composition from DHA. This previously unidentified peptido-lipid has potent bioactivity and significantly enhances planaria regeneration. Additionally, this peptido-lipid significantly delays the formation of granulomas by human peripheral blood mononuclear cells. We also disclose analogs of this composition that can be used in treating a variety of conditions as discussed further below.

### SUMMARY OF THE INVENTION

The inventors have recently identified a family of conjugated molecules having lipid and peptide moieties in self-resolving inflammatory exudates. The compounds have a lipid backbone and give UV chromophores characteristic of a conjugated triene double bond system coupled to an auxochrome allylic to the triene. Further elucidation of the compounds reveals that they have a lipid backbone conjugated to an amino acid or peptide moiety via an auxochrome. In some cases, the auxochrome is sulfur. However, the auxochrome may be NH, CH₂ or O. The compounds have potent bioactivity, in vitro and, in vivo, including reduction or prevention of granuloma formation, increasing tissue regeneration, promoting resolution of infection, stimulating macrophage phagocytosis of bacteria; protecting tissues from neutrophil mediated damage.

Therefore, in various exemplary embodiments, the invention includes, a purified compound comprising: an SPM or SPM derivative conjugated at carbon 4 or 5 by an auxochrome to an amino acid, peptide or peptide derivative or a pharmaceutically acceptable salt of the conjugate compound. In some embodiments, the SPM is derived from docosahexaenoic acid. In various exemplary embodiments, the auxochrome is: S, NH, CH₂, or O.

In various embodiments the amino acid or peptide derivative is glutathione.

In still other exemplary embodiments, this disclosure provides a compound having a general formula I and Ia.

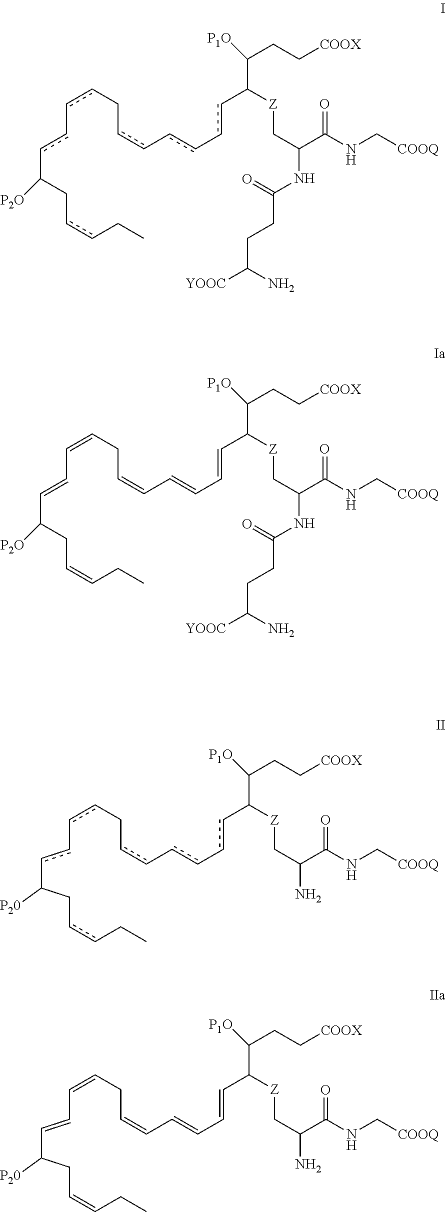

In these exemplary embodiments, P₁ and P₂ individually are a protecting group or a hydrogen atom; [symbol] is a double bond; each double bond is independently in the E or Z configuration; each Q is independently H, Me, Et, iPr, or —CF3; each X is independently H, Me, Et, iPr, —CF3, each Y is independently H, Me, Et, iPr, —CF3; and Z is independently S, NH, CH₂, or O; optionally when Q, X, and Y are H then P₁ and P₂ cannot both be H if Z is S; or a pharmaceutically acceptable salt or ester thereof.

In some embodiments, the compounds are 4,5-RCTR1:

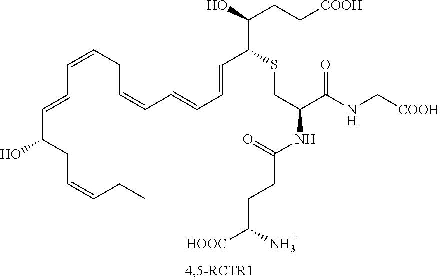 [SMILES: CC/C=C\C[C@H](O)/C=C/C=C\C\C=C/C=C/C=C/[C@@H](SC[C@H](NC(=O)CC[C@H](N)C(=O)O)C(=O)NCC(=O)O)[C@@H](O)CCC(=O)O]

or a pharmaceutically acceptable salt or ester thereof.

In yet other exemplary embodiments, the compound is:

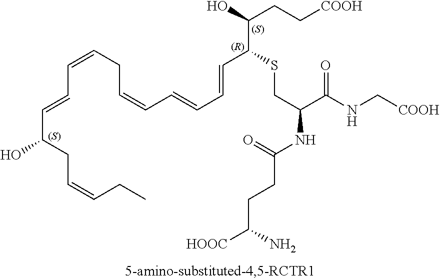

or a pharmaceutically acceptable salt or ester thereof.

In still other exemplary embodiments, the compound is:

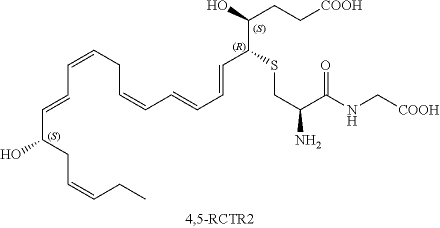

or a pharmaceutically acceptable salt or ester thereof.

In various other exemplary embodiments, the compounds is:

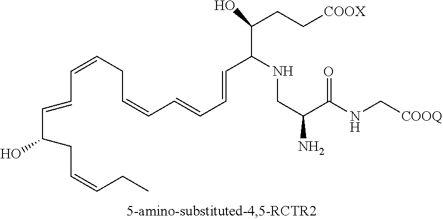

or a pharmaceutically acceptable salt or ester thereof.

In some embodiments, the compounds are purified.

In various embodiments, a composition comprising any of the compounds and a pharmaceutically acceptable carrier is disclosed.

In still other exemplary embodiments also disclosed is a method of treating inflammation or an inflammatory disease comprising administering to a subject in need thereof a compound or composition of any of the compounds of the invention.

In still yet other exemplary embodiments, the invention provides a method of enhancing tissue repair or tissue regeneration comprising, administering to a subject in need thereof a compound or composition of any of the compounds of the invention. In these embodiments, enhancing tissue repair or tissue regeneration comprises preventing or ameliorating second organ reperfusion injury.

In yet other embodiments, disclosed herein is a method of delaying, preventing or treating diseases of granuloma formation comprising, administering to a subject in need thereof a compound or composition any of the compounds of the invention. In some embodiments, diseases of granuloma formation comprise Mycobacterium tuberculosis, chronic granulomatous disease, granulomatous steatitis, foreign-body granulomas, interstitial lung fibrosis (ipf), leprosy, arthritis, sarcoidosis, liver fibrosis, heart fibrosis, renal fibrosis, hepatic cirrhosis, pulmonary fibrosis and organ fibrosis.

In still other exemplary embodiments, disclosed is a method of enhancing resolution of inflammatory diseases wherein the disease comprises: preventing or treating granuloma formation in a subject in need there of comprising administering a compound or composition of any of the compounds of the invention. In some embodiments, inflammatory disease comprise oral inflammation, periodontitis, ulcerative colitis, Cohn's disease, arthritis, asthma and chronic obstructive pulmonary disease, hepatitis, sinusitis, systemic lupus, allergies, dermatitis, atherosclerosis, psoriasis, bronchitis, appendicitis, neurodegenerative diseases, and multiple sclerosis.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE FIGURES

Various exemplary embodiments of the compositions and methods according to the invention will be described in detail, with reference to the following figures wherein:

FIG. 1: Identification of Novel Peptido-Lipid from 4S, 5S-epoxy-Resolvin. Human macrophages, PMNs, and recombinant enzymes were incubated with 4S, 5S-epoxy-resolvin. A) LC-MS/MS MRM monitoring in positive ion mode for m/z 666>225. B) MS/MS fragmentation spectrum with proposed ions. C) Theoretical structure with proposed fragmentations.

FIG. 2: Regenerative Action of 4,5-RCTR1 on Planaria. Planaria were surgically injured post-ocular and received either vehicle (0.1% ethanol) or 10 nM 4,5-RCTR1 one-hour post-injury and imaged daily for six days. A) Representative images of Planaria regeneration from Day 1-6 post-surgical injury. B) Tissue regeneration indices (μm) from Day 1-6 post-surgically injured Planaria. C) Tissue regeneration rate (TRR, μm/day) is the rate (area of tissue regeneration/days at TRI₅₀) to achieve 50% tissue regeneration after surgical injury. n=6 Planaria per group per day. Data represented as mean±SEM. Unpaired two-tailed Student's t-test. *p<0.05, **p<0.01, ***p<0.001, and ****p<0.0001 versus vehicle.

FIG. 3: Resolvin (Rv) Peptido-Lipids Delay Human Granuloma Formation. Freshly isolated human PBMCs were incubated with E. coli total protein extract covalently coupled CNBr-activated sepharose beads and received a single treatment of either vehicle (0.1% methanol or ethanol), 10 nM RvD3, 10 nM RCTR1, or 10 nM 4,5-RCTR1 at time 0 and incubated for 6 days. A) Scoring of day 3 granuloma severity post-single treatment with either vehicle (0.1% methanol or ethanol), 10 nM RvD3, 10 nM RCTR1, or 10 nM 4,5-RCTR1. n=3 individual human donors. Mean±SEM. Unpaired two-tailed Student's t-test. **p<0.01, ***p<0.001 versus vehicle. B) Representative images of granuloma formation on day 3 after a single treatment with either vehicle (0.1% methanol or ethanol), 10 nM RvD3, 10 nM RCTR1, or 10 nM 4,5-RCTR1. Magnification, 40×. Scale bar=30 μm.

FIG. 4: 4S, 5S-epoxy-Resolvin Precursor to Novel Bioactive Mediators. Proposed biosynthetic scheme and functions of each of the resolvins (RvD3, 4,5-RCTR1, and RvD4).

FIG. 5: NMR Validation of 4S, 5S-epoxy-Resolvin from Total Organic Synthesis. A) UV chromophore for 4S, 5S-epoxy-17S-hydroxy-resolvin methyl ester with λₘₐₓ^{MeOH}=281 nm for the conjugated triene and 228 nm for the diene moieties, respectively. B) Structure of 4S, 5S-epoxy-17S-hydroxy-resolvin methyl ester denoting hydrogens detected by two-dimensional COSY NMR. C) Two-dimensional COSY NMR of 4S, 5S-epoxy-17S-hydroxy-resolvin methyl ester. Double-bond geometry was assigned using 2D 1H-¹H NMR on a Varian NMRS 600 MHz NMR spectrometer at 25° C. on a 5 mm Triple Resonance PFG¹H probe and referenced to the benzene-d₆ (C₆D₆) internal standard. The rainbow plot depicts positive contours of cross peaks along the diagonal axis allowing for the full proton assignment. The zoomed-in region highlights the Z/E olefinic protons H₆-H₁₁, H₁₃-H₁₆ and H₁₉-H₂₀.

FIG. 6: Human Neutrophils (PMN) Biosynthesize RvD3 and RvD4 from 4S, 5S-epoxy-Resolvin. The 4S, 5S-epoxy-resolvin (1 μg) was incubated for 5 min at 37° C. in PBS⁺⁺ (pH 7.4) to obtain the aqueous hydrolysis profile for direct comparisons. A) LC-MS/MS MRM monitoring in negative ion mode for m/z 375>147. B) Structure and MS/MS fragmentation spectrum with proposed ions matching the non-enzymatic hydrolysis products I (left) and II (right). C) Structure and MS/MS fragmentation spectrum with proposed ions matching the non-enzymatic hydrolysis products III (left) and IV (right). D) Human neutrophils (1×10⁶) were incubated with 4S, 5S-epoxy-resolvin (500 ng) for 15 min at 37° C. in 500 μL PBS⁺⁺ (pH 7.4). (left) LC-MS/MS MRM monitoring in negative ion mode for m/z 375>147 (RvD3). (right) Structure and MS/MS fragmentation spectrum with proposed ions matching RvD3. E) (left) LC-MS/MS MRM monitoring in negative ion mode for m/z 375>101 (RvD4) and structure. (right) MS/MS fragmentation spectrum with proposed ions matching RvD4.

FIG. 7: Human Neutrophils Express Glutathione S-Transferases and Biosynthesize Novel Peptido-Lipid Mediator from 4S, 5S-epoxy-Resolvin. Human neutrophils were isolated from peripheral blood of donors and subjected to flow cytometry. Debris and doublets were first gated out before further gating on CD66b followed by LTC₄S, mGST2, mGST3, and GSTM4. A) (left) Representative gating depicting the unstained cell population (black) and CD6611⁺ (blue). (right) Representative histogram of LTC₄S and mGST3 expression. B) Mean fluorescent intensity (MFI) of LTC₄S, mGST2, mGST3, and GSTM4 in CD66b⁺ cells. n=4 individual human donors. Mean±SEM. Ordinary One-way ANOVA with Tukey multiple comparisons test. *p<0.05 and **p<0.01 versus secondary only. C) Human neutrophils (1×10⁶) were incubated with 4S, 5S-epoxy-resolvin (500 ng) for 15 min at 37° C. in 500 μL PBS⁺⁺ (pH 7.4). Structure and MS/MS fragmentation spectrum with proposed ions matching Product 1.

FIG. 8: Human Neutrophils Biosynthesize Cysteinyl-Leukotrienes from LTA₄. Human neutrophils (1×10⁶) were incubated with leukotriene A₄ (500 ng) for 15 min at 37° C. in 500 μL PBS⁺⁺ (pH 7.4). A) (left) Structure and LC-MS/MS MRM monitoring in negative ion mode for m/z 335>195 (LTB₄) (right) MS/MS fragmentation spectrum with proposed ions matching LTB₄. B) (left) Structure and LC-MS/MS MRM monitoring in positive ion mode for m/z 626>189 (LTC₄) (right) MS/MS fragmentation spectrum with proposed ions matching LTC₄. C) (left) Structure and LC-MS/MS MRM monitoring in positive ion mode for m/z 497>189 (LTD₄) (right) MS/MS fragmentation spectrum with proposed ions matching LTD₄. D) (left) Structure and LC-MS/MS MRM monitoring in positive ion mode for m/z 440>189 (LTE₄) (right) MS/MS fragmentation spectrum with proposed ions matching LTE₄.

FIG. 9: Human Macrophages Biosynthesize Novel Peptido-Lipid Mediator (Product 1) from 4S, 5S-epoxy-Resolvin. Human macrophages (10⁷ cells/mL) were incubated with 4S, 5S-epoxy-resolvin (1 μg) for 5 min at 37° C. in PBS⁺⁺ (pH 7.4). A) LC-MS/MS MRM monitoring in positive ion mode for m/z 666>648 (Product 1).

FIG. 10: Human Macrophages Biosynthesize Novel Peptido-Lipid Mediator (Product 2) from 4S, 5S-epoxy-Resolvin. Human macrophages (10⁷ cells/mL) were incubated with 4S, 5S-epoxy-resolvin (1 μg) for 5 min at 37° C. in PBS⁺⁺ (pH 7.4). A) LC-MS/MS MRM monitoring in positive ion mode for m/z 537>501 (Product 2) and theoretical structure. B) MS/MS fragmentation spectrum with proposed ions matching Product 2. C) Proposed biosynthetic pathway of Product 1 and Product 2.

FIG. 11: Structural Similarity of Novel Lipid 4S, 5S-epoxy-resolvin Peptido-Lipids to RCTR1 and RCTR2. Authentic total organic synthesized standards for RCTR1 and RCTR2. A) (left) LC-MS/MS MRM monitoring in positive ion mode for m/z 666>630 (RCTR1) and structure. (right) MS/MS fragmentation spectrum with proposed ions matching RCTR1. B) (left) LC-MS/MS MRM monitoring in positive ion mode for m/z 537>501 (RCTR2) and structure. (right) MS/MS fragmentation spectrum with proposed ions matching RCTR2.

FIG. 12: Recombinant Human Enzymatic Conjugation of 4S, 5S-epoxy-resolvin to Product 1. Human recombinant LTC₄S (2 μg), mGST2 (2 μg), mGST3 (2 μg), and GSTM4 (2 μg) were incubated with 4S, 5S-epoxy-resolvin (100 ng) and reduced glutathione (5 mM) in 25 mM Tris-HCl (pH 8.0), 0.05% Triton X-100 for 15 min at 37° C. A) The percent conversion of 4S, 5S-epoxy-resolvin to Product 1 by LTC₄S, mGST2, mGST3, and GSTM4. B) Scheme of SN2 reaction of reduced glutathione and 4S, 5S-epoxy-resolvin catalyzed by LTC₄S. C) LC-MS/MS MRM monitoring in positive ion mode for m/z 666>648 (Product 1) and structure. D) MS/MS fragmentation spectrum with proposed ions matching Product 1.

FIG. 13: 3D structure of 4,5-RTCR1 confirmed by endogenous biosynthesis and spatial filling.

### DETAILED DESCRIPTION OF THE EXEMPLARY EMBODIMENTS

The acute inflammatory response is essential for host defense and requires active resolution to return to homeostasis. Failed resolution plays a major role in the development of chronic inflammation and the etiology of multiple diseases such as arthritis, periodontitis, thrombosis, fibrosis, and cancer. The resolution of inflammation is an active process driven by specialized pro-resolving lipid mediators (SPM) that arise from polyunsaturated fatty acids such as arachidonic acid, docosahexaenoic acid, eicosapentaenoic acid, and docosapentaenoic acid. SPMs counter-regulate pro-inflammatory mediators, increase uptake of bacteria, cellular debris, and apoptotic neutrophils, and decrease the influx of infiltrating leukocytes. Granuloma formation occurs during inflammation when the immune system is unable to eliminate bacteria, fungi, or a foreign body and instead forms an enclosure. Granulomas are the basis for multiple infectious and non-infectious diseases such as Tuberculosis, Leprosy, Crohn's disease, Rheumatoid arthritis, Sarcoidosis, Chronic granulomatous disease, foreign-body granulomas, and aspiration pneumonia. Per the World Health Organization, ˜10 million people had Tuberculosis worldwide in 2018.

Abbreviations used throughout the specification:
AA, arachidonic acid;
CFU, colony forming unit;
EFA, essential fatty acid;
DHA, 4Z, 7Z,10Z,13Z,16Z,19Z-docosahexaenoic acid;
GC-MS, gas chromatography-mass spectrometry;
GGT, γ-glutamyl transferase;
HpD, hydro(peroxy)-docosahexaenoic acid;
LC/MS/MS, liquid chromatography-tandem mass spectrometry;
LM, lipid mediator;
LT, leukotriene;
LO, lipoxygenase;
MRM, multiple reaction monitoring;
MS, mass spectrometry; m/z, mass-to-charge ratio;
PG, prostaglandin;
PMN, polymorphonuclear neutrophil;
RCTR, resolvin conjugate in tissue regeneration;
4,5-RCTR1,5-glutathionyl, 4,17-hydroxy-docosahexaenoic acid;
SPM, specialized proresolving mediator;
T50, time to 50% regeneration;
TR, retention time;
TRI, tissue regeneration index;
TRImax, maximum tissue regeneration;
MΦ, macrophage;
PGE₂, prostaglandin E2;
PMN, polymorphonuclear neutrophils;
Rv, resolvin;
RvD1, Resolvin D1, 7S,8R,17S trihydroxydocosa-4Z,9E,11E,13Z,15E,19Z-hexaenoic acid;
RvD2, Resolvin D2, 7S,16R,17S-trihydroxy-4Z,8E,10Z,12E,14E,19Z-docosahexaenoic acid;
RvD3, Resolvin D3, 4S,11R,17S-trihydroxy-5Z,7E,9E,13Z,15E,19Z-docosahexaenoic acid;
RvD4, Resolvin D4, (4S,5R,6E,8E,10Z,13Z,15E,17S,19Z)-4,5,17-trihydroxy-6,8,10,13,15,19-docosahexaenoic acid;
SPM, specialized proresolving mediator

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by”, “contain(s)” and “having” and variants thereof can be used interchangeably and are intended to be open-ended transitional phrases, terms, or words that do not preclude the possibility of additional acts or structures.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

“Compounds of the invention” refers to the bioactive peptide conjugates of DHA, analogues and compounds encompassed by generic formulae disclosed herein and includes any specific compounds within those formulae whose structure is disclosed herein. The compounds of the invention may be identified either by their chemical structure and/or chemical name. When the chemical structure and chemical name conflict, the chemical structure is determinative of the identity of the compound. The compounds of the invention may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention also include isotopically labeled compounds where one or more atoms have an atomic mass different from the atomic mass conventionally found in nature.

The compounds depicted throughout the specification contain ethylenic unsaturated sites. Where carbon-carbon double bonds exist, the configurational chemistry can be either cis (Z) or trans (E) and the depictions throughout the specification are not meant to be limiting. The depictions are, in general, presented based upon the configurational chemistry of DHA compounds, and although not to be limited by theory, are believed to possess similar configuration chemistry. The use of [symbol] reflects this throughout the specification and claims so that both cis (Z) and trans (E) isomers are contemplated. In certain embodiments the configuration of the ethylenic bond is known and is particularly described.

In one aspect of the invention, the compound(s) of the invention are substantially purified and/or isolated by techniques known in the art. The purity of the purified compounds is generally at least about 50%, preferably 90%, more preferably at least about 95%, and most preferably at least about 99% by weight.

Thus, the term “purified” as used herein does not require absolute purity; rather, it is intended as a relative term. For example, a purified DHA analogue can be one in which the subject DHA analogue is at a higher concentration than the analogue would be in its natural environment within an organism. For example, a DHA or EPA analogue of the invention can be considered purified if the analogue content in the preparation represents at least 10%, 50%, 60%, 70%, 80%, 85%, 90%, 92%, 95%, 98%, or 99% of the total analogue content of the preparation. Artisans will immediately appreciate that all the ranges and values within the explicitly stated ranges are contemplated.

“Auxochrome” as used herein refers to refers to an atom or molecule or moiety of a molecule that influences the intensity of absorption of the molecule.

“Biological activity” and its contextual equivalents “activity” and “bioactivity” means that a compound elicits a statistically valid effect in any one biological test assays. Preferably, the threshold for defining an “active” compound will be reproducible and statistically valid effects of at least 25% deviation from untreated control at concentrations at or lower than 1 μM.

“Biological test assay” means a specific experimental procedure. Non-limiting examples of biological test assays include: 1) ligand binding, either direct or indirect, to a purified target, subcellular fraction, intact cell, or cell or tissue extract; 2) metabolic protection with enhanced half-life when exposed to a purified target, subcellular fraction, intact cell, cell or tissue extract, or administered to intact organism by any route; 3) prevention, reversal, or amelioration of cell- and tissue-based functional responses recognized by skilled artisans to represent surrogates for anti-inflammatory action (e.g., altered cytokine production and release); and 4) prevention, reversal, or amelioration of symptoms and/or disease processes in animal models of inflammation and inflammatory disease.

“Conjugated” when used herein in its broadest sense such as “conjugated compounds” two or more joined compounds or moieties. The compounds can be joined by a bond such as by covalent bonds or non-covalent bonds including, ionic bonds, hydrogen bonds, van der Waals forces and the like. “Conjugated” may be used in a more specific sense as well, as in a “conjugated bond system” meaning a system of connected p-orbitals with delocalized electrons in compounds with alternating single and multiple bonds, which in general may lower the overall energy of the molecule and increase stability. Lone pairs, radicals or carbenium ions may be part of the system. The compound may be cyclic, acyclic, linear or mixed.

“Triene” as used herein means a conjugated bond system including three double bonds.

“Detectable label” means any chemical or biological modality which can be used to track, trace, localize, quantify, immobilize, purify, or identify compounds through appropriate means of detection known in the art. Non-limiting examples of detectable labels include fluorescence, phosphorescence, luminescence, radioactive or biospecific affinity capture labels.

“Derivative” as used herein means derived from. For example, a DHA analogue may be derived from DHA. The derivative may be a natural metabolite, or it may be a synthetic compound derived from a natural compound or the compound may be synthesized in toto.

“Electronegative group” is a chemical group that tends to acquire rather than lose electrons in its chemical interactions. Examples of electronegative groups include, but are not limited to, —NO₂, ammonium salts, sulfonyl groups, carbonyl groups, halogens, esters, carboxylic acids, nitriles, etc.

“In Situ” refers to and includes the terms “in vivo,” “ex vivo” and “in vitro” as these terms are commonly recognized and understood by the skilled artisan. Moreover, the phrase “in situ” is employed herein in its broadest connotative and denotative context to identify an entity, cell, or tissue as found or in place, without regard to its source or origin, its condition or status or its duration or longevity at that location or position.

“Moiety” as used herein refers to specific groups of atoms or bonds within molecules that are responsible for the characteristic chemical reactions of those molecules.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) salts fanned when an basic proton is present in the parent compound such as acid addition salts, formed with inorganic acids such as hydrochloric acid, hydrobromic acid, sulfuric acid, nitric acid, phosphoric acid, and the like; or those formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton is present in the parent compound and either is replaced by a metal ion, e.g., an alkali metal ion, an alkaline earth ion, or an aluminum ion; or coordinates with an organic base such as ethanolamine, diethanolamine, triethanolamine, N-methylglucamine, triethylamine, propylamino, diazabicycloundecane and the like.

“Pharmaceutically acceptable vehicle” refers to a diluent, adjuvant, excipient or carrier with which a compound of the invention is administered.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Prodrug” refers to a derivative of a drug molecule that requires a transformation within the body to release the active drug. Prodrugs are frequently (though not necessarily) pharmacologically inactive until converted to the parent drug. A hydroxyl containing drug may be converted to, for example, to a sulfonate, ester or carbonate prodrug, which may be hydrolyzed in vivo to provide the hydroxyl compound. An amino containing drug may be converted, for example, to a carbamate, amide, imine, phosphonyl, phosphoryl or sulfenyl prodrug, which may be hydrolyzed in vivo to provide the amino compound. A carboxylic acid drug may be converted to an ester (including silyl esters and thioesters), amide or hydrazide prodrug, which be hydrolyzed in vivo to provide the carboxylic acid compound. Prodrugs for drugs which contain different functional groups other than those listed above are well known to the skilled artisan.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2.sup.nd ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated (e.g., methyl and ethyl esters, acetate or propionate groups or glycol esters) or alkylated such as benzyl, and trityl ethers as well as alkyl ethers, tetrahydropyranyl ethers, trialkylsilyl ethers (e.g., TMS or TIPPS groups) and allyl ethers.

“Subject” means living organisms susceptible to conditions or diseases caused or contributed to by infection, inflammation, inflammatory responses, vasoconstriction and myeloid suppression. Examples of subjects include humans, dogs, cats, cows, goats and mice. The term subject is further intended to include transgenic species such as, for example, transgenic mice.

The present invention is also drawn to methods for treating or preventing arterial granuloma formation in inflammation, arthritis, psoriasis, urticaria, vasculitis, asthma, ocular inflammation, pulmonary inflammation, pulmonary fibrosis, oral inflammation, periodontitis, ulcerative colitis, Crohn's disease, arthritis, asthma and chronic obstructive pulmonary disease, hepatitis, sinusitis, systemic lupus, allergies, dermatitis, atherosclerosis, psoriasis, bronchitis, appendicitis, neurodegenerative diseases, and multiple sclerosis, in a subject by administration of one or more of the DHA conjugates described herein. Disease states or conditions that are associated with granuloma formation due to inflammation such as the recruitment of neutrophils, leukocytes and/or cytokines are included within the general scope of inflammation and include, for example, allergy, Alzheimer's disease, arthritis, asthma, atherosclerosis, cancer, colon cancer, degenerative neurologic disorders, dementia, dermatology, diabetes mellitus, lung cancer, Mycobacterium tuberculosis, chronic granulomatous disease, granulomatous steatitis, foreign-body granulomas, interstitial lung fibrosis (ipf), leprosy, sarcoidosis, liver fibrosis, heart fibrosis, renal fibrosis, hepatic cirrhosis, pulmonary fibrosis, and organ fibrosis.

The pharmaceutical compositions of the invention include a “therapeutically effective amount” or a “prophylactically effective amount” of one or more of the DHA analogs of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the DHA analog may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the DHA analog and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a DHA analog of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one DHA analog, in combination with a pharmaceutically acceptable carrier.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively nontoxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as nontoxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1 19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, acetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention. Such solutions are useful for the treatment of conjunctivitis.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about 20 micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The invention features an article of manufacture that contains packaging material and DHA analog formulation contained within the packaging material. This formulation contains an at least one DHA analog and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable DHA analogs are described herein.

When the body is unable to contain infections, this may lead to collateral organ damage resulting from unchecked innate immune responses. Here the inventors investigated the chemical signals produced by immune cells to expedite clearance of bacteria, promote organ repair and tissue regeneration. The inventors identified molecules produced during self-limited infections and in human milk that promote clearance of bacteria as well as accelerate tissue regeneration. In addition, these molecules also protected organs from exuberant inflammatory responses, such as, ischemia/reperfusion injury, by limiting select white blood cell recruitment and upregulating the expression of proteins involved in tissue repair. Therefore, these results identify new resolution moduli that regulate phagocytes to clear bacteria, activate the regeneration milieu.

### EXAMPLES

Various exemplary embodiments of devices and compounds as generally described above and methods according to this invention, will be understood more readily by reference to the following examples, which are provided by way of illustration and are not intended to be limiting of the invention in any fashion.

### Example 1

### Identification of Novel Peptido-Lipid from 4S, 5S-epoxy-Resolvin

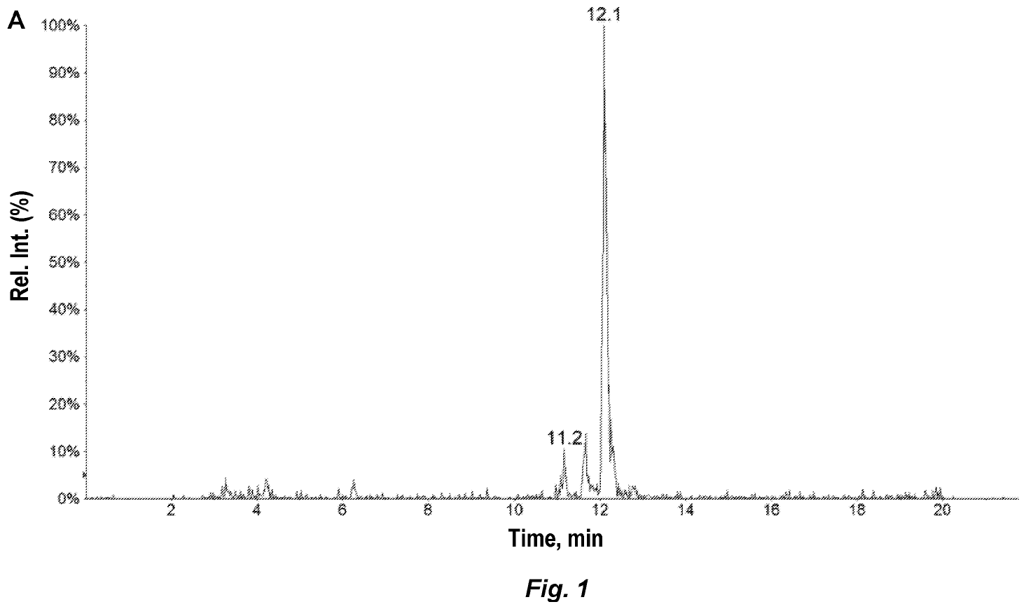

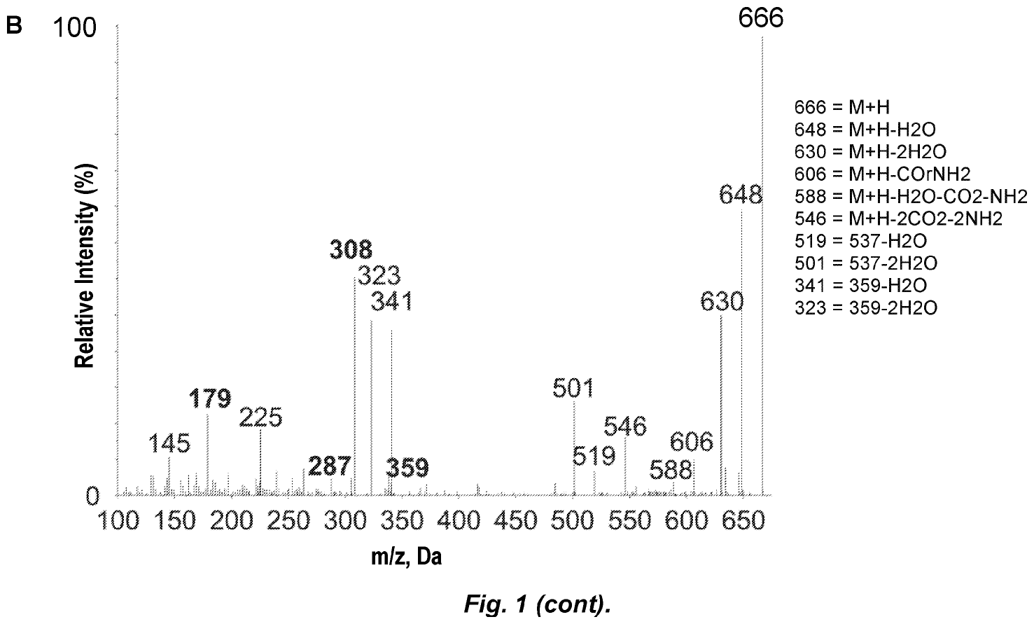

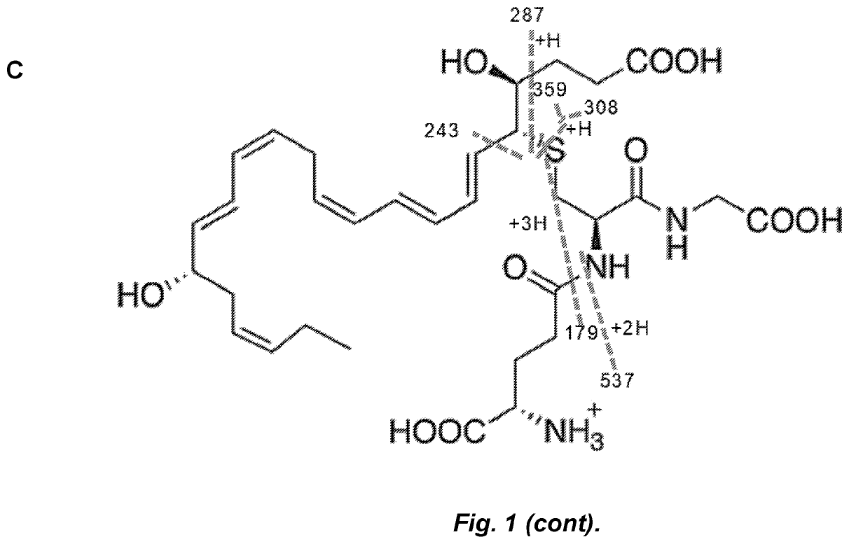

Human macrophages, PMNs, and recombinant enzymes were incubated with 4S, 5S-epoxy-resolvin. FIGS. 1A, 1B and 1C. 1A) LC-MS/MS MRM monitoring in positive ion mode for m/z 666>225. 1B) MS/MS fragmentation spectrum with proposed ions. 1C) Theoretical structure with proposed fragmentations.

### Example 2

### Planaria Regeneration

Planaria (Dugesia japonica) were kept in water (Poland Spring, Poland, ME) at 20° c. All animals were starved for 7 days prior to surgery. Planaria were surgically injured post-ocularly and received either vehicle (0.1% ethanol) or 10 nM 4,5-RCTR1 in water one-hour post-injury. Tissue regeneration was monitored daily for six days via a Zeiss Axiovert 40 CFL Microscope (Jena, Germany) equipped with a Lumenera Infinity 3 Camera (Ottawa, Canada) running Image-Pro Plus (Version 7.0.0.591, Media Cybernetics, Rockville, MD, USA). Regenerated tissue was quantified using ImageJ software (NIH). The tissue regeneration index (TRI) normalizes the total area of regenerated tissue by the post-ocular width. The TRI₅₀ is the amount of time it takes to reach 50% of the maximum number of neutrophils.

### Example 3

### Regenerative Action of 4,5-RCTR1 on Planaria

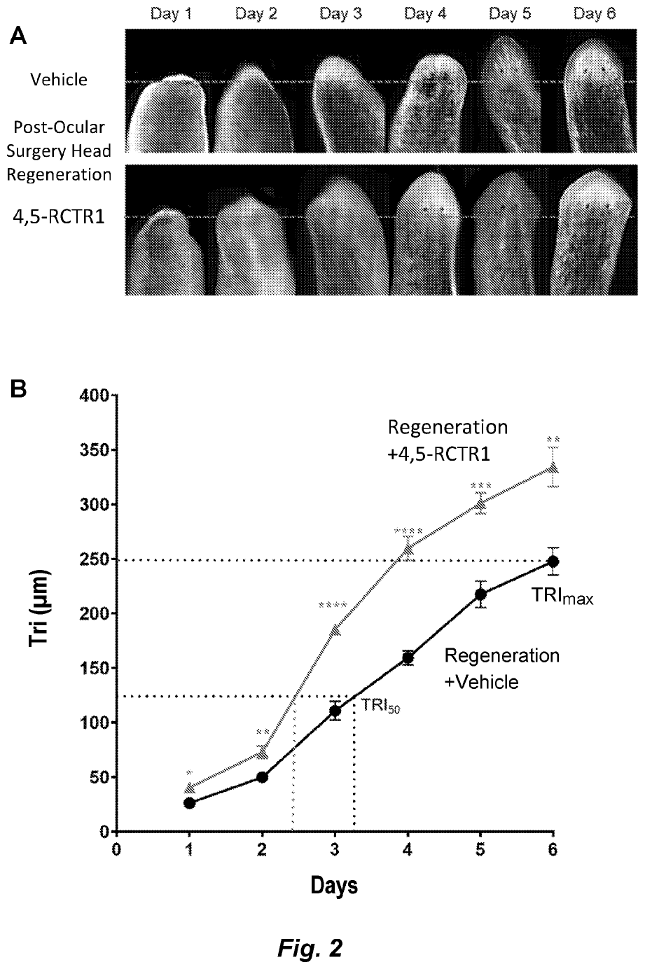

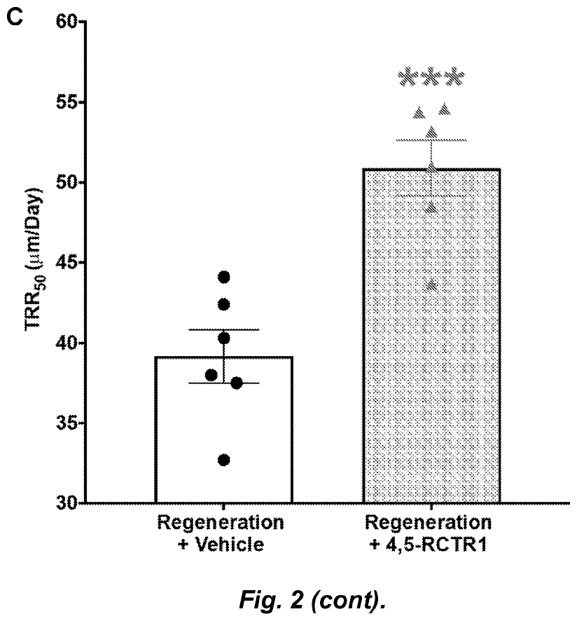

4,5-RCTR1 was shown to have regenerative action. FIGS. 2A, 2B and 2C: Planaria were surgically injured post-ocular and received either vehicle (0.1% ethanol) or 4,5-RCTR1 one-hour post-injury and imaged daily for six days. 2A) Representative images of Planaria regeneration from Day 1-6 post-surgical injury. 2B) Tissue regeneration indices (μm) from Day 1-6 post-surgically injured Planaria. 2C) Tissue regeneration rate (TRR, μm/day) is the rate (area of tissue regeneration/days at TRI₅₀) to achieve 50% tissue regeneration after surgical injury. n=6 Planaria per group per day. Data represented as mean±SEM. Unpaired two-tailed Student's t-test. *p<0.05, **p<0.01, ***p<0.001, and ****p<0.0001 versus vehicle.

### Example 4

### Granuloma Assay

E. coli (Serotype: 06:K2:H1) was grown overnight in Luria broth in an orbital shaker (400 rpm) at 37° C. To obtain a total protein extract, E. coli cells were lysed in coupling buffer (0.1M NaHCO₃, pH 8.3+0.5M NaCl+1X protease inhibitors cocktail (Thermo Fisher Scientific, Waltham, MA, USA)) through multiple freeze-thaw cycles and pellet disruption. Preparation of sepharose and coupling occurred as per the manufacturer's instructions. Briefly, 38 mg lyophilized CNBr-activated Sepharose 4B beads (Sigma-Aldrich) were washed with 200 mL 1 mM HCl, pH 3.0 (added in several aliquots) on a sintered glass filter. The beads were rotated at room temperature for one hour with 3 mL coupling buffer (0.1M NaHCO₃, pH 8.3+0.5M NaCl)+1X protease inhibitors cocktail (Thermo Fisher Scientific))+763 μg total E. coli protein extract. Supernatant was taken before and after the coupling reaction and measured for protein content by BCA assay (Thermo Fisher Scientific) to determine the efficiency of the reaction. Excess ligand was washed away with 5 mL of coupling buffer. Any remaining active groups were blocked with 0.1M Tris-HCl, pH 8.0 for 2 hours. The medium was washed with 5 mL of 0.1M acetic acid/sodium acetate, pH 4.0⁺0.5M NaCl followed by a wash with 0.1M Tris-HCl, pH 8.0+0.5M NaCl and repeated three times.

Freshly isolated PBMCs (1×10⁶ cells/well) were treated with either vehicle (0.1% ethanol), RvD3 (10 nM), RCTR1 (10 nM) or 4,5-RCTR1 (10 nM) and E. coli total protein lysate-coated CNBr-activated sepharose beads simultaneously in 1 mL complete RPMI-1640. Granulomas were imaged with a Zeiss Axiovert 40 CFL Microscope equipped with a Lumenera Infinity 3 Camera running Image-Pro Plus (Version 7.0.0.591) from days 1 through 6 of formation. Granulomas were scored based on formation progression. Scoring ranged from 0-4; 0: No progression. 1: One or more cells adhering to the bead. 2: Formation of a monolayer. 3: Formation of multiple layers. 4: Multiple adherent layers accompanied by mononuclear cell migration and giant cell formation. (Shikama 1989 AJP, and Puissegur 2004 Cellular Microbiology)

### Example 5

### Resolvin (Rv) Peptido-Lipids Delay Human Granuloma Formation

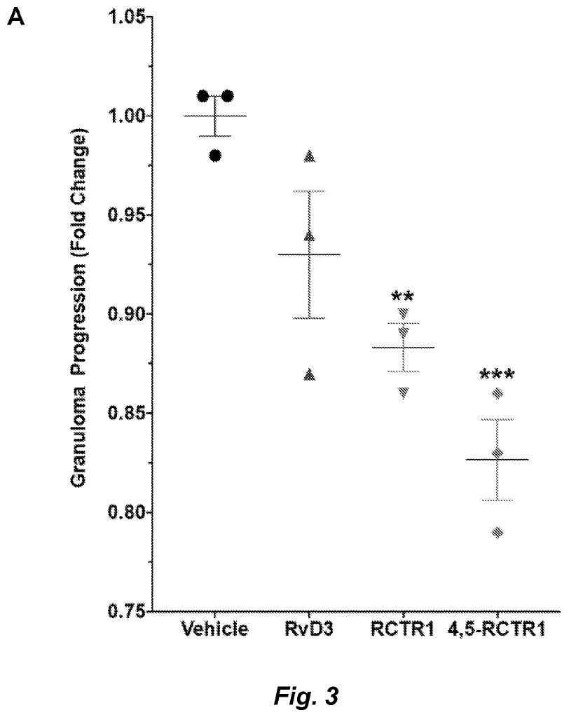

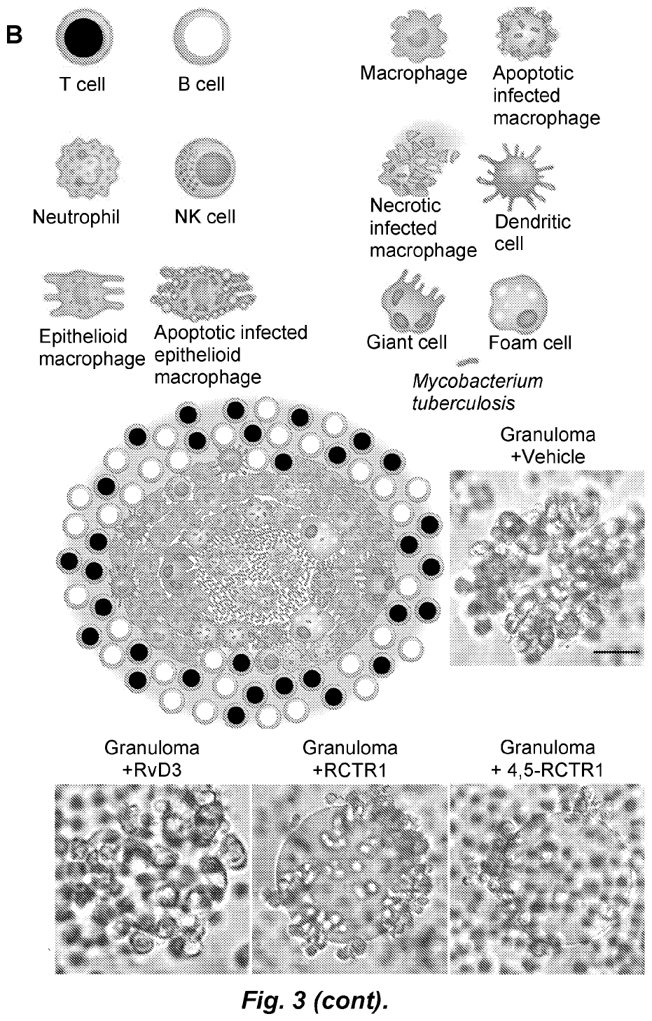

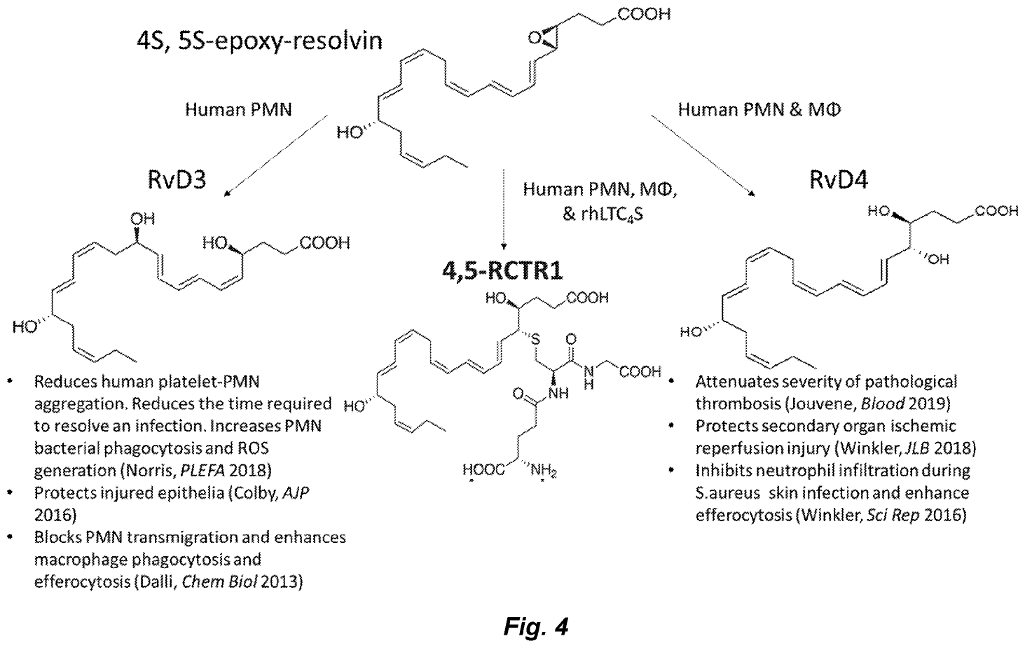

Freshly isolated human PBMCs were incubated with E. coli total protein extract covalently coupled CNBr-activated sepharose beads and received a single treatment of either vehicle (0.1% methanol or ethanol), 10 nM RvD3, 10 nM RCTR1, or 10 nM 4,5-RCTR1 at time 0 and incubated for 6 days. FIGS. 3A, and 3B: 3A) Scoring of day 3 granuloma severity post-single treatment with either vehicle (0.1% methanol or ethanol), 10 nM RvD3, 10 nM RCTR1, or 10 nM 4,5-RCTR1. n=3 individual human donors. Mean±SEM. Unpaired two-tailed Student's t-test. **p<0.01, ***p<0.001 versus vehicle. 3B) Representative images of granuloma formation on day 3 after a single treatment with either vehicle (0.1% methanol or ethanol), 10 nM RvD3, 10 nM RCTR1, or 10 nM 4,5-RCTR1. Magnification, 40×. Scale bar=30 μm.

### Example 6

### 4S, 5S-epoxy-Resolvin Precursor to Novel Bioactive Mediators

Proposed biosynthetic scheme and functions of each of the resolvins (RvD3, 4,5-RCTR1, and RvD4)

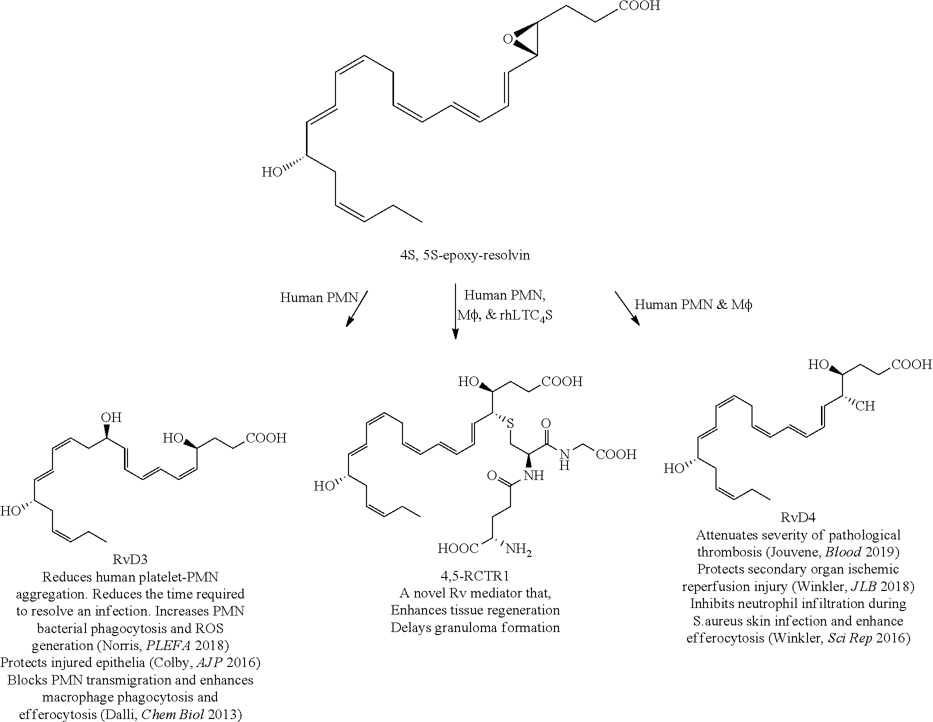

### Example 7

### Resolvin Pathway through 4S, 5S-epoxy-17S-hydroxy-intermediate (4S,5S-epoxy-resolvin)

The Resolvin D3 (RvD3) and Resolvin D4 (RvD4) biosynthesis from docosahexaenoic acid (DHA) through a 4S, 5S-epoxy-17S-hydroxy-intermediate (4S,5S-epoxy-resolvin) were established. The complete stereochemistries of each were established by our laboratory and are shown in Scheme 1, below.

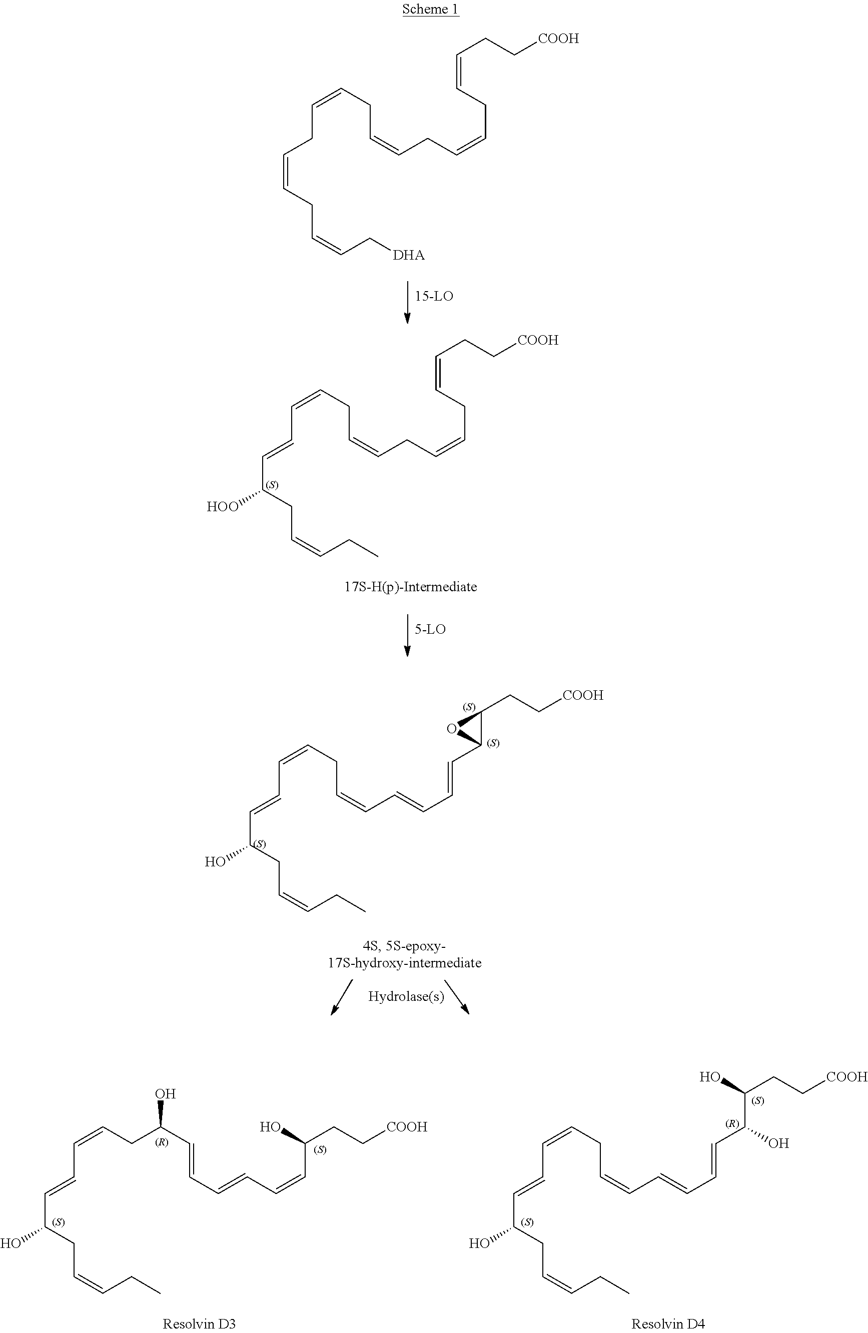

### Example 8

### NMR Validation of 4S, 5S-epoxy-Resolvin from Total Organic Synthesis

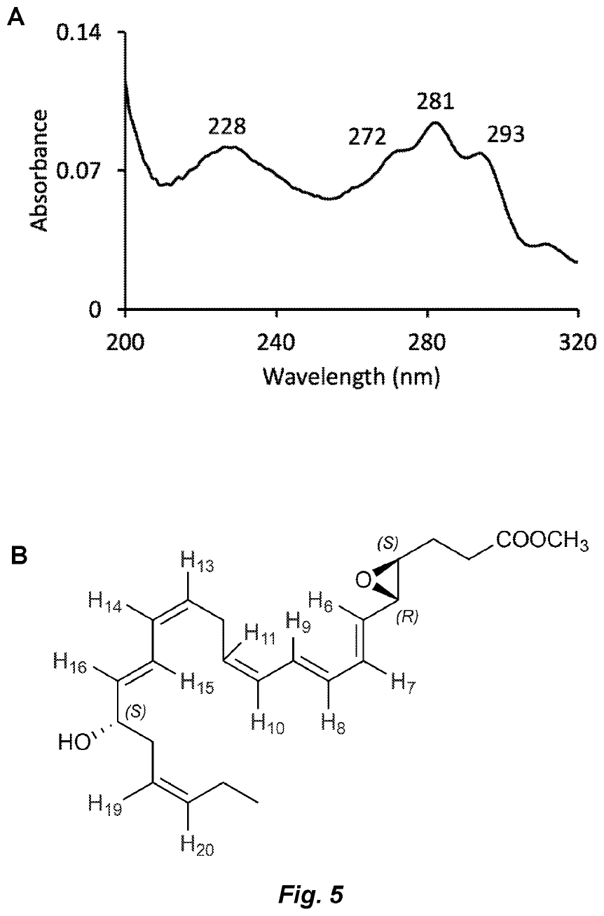

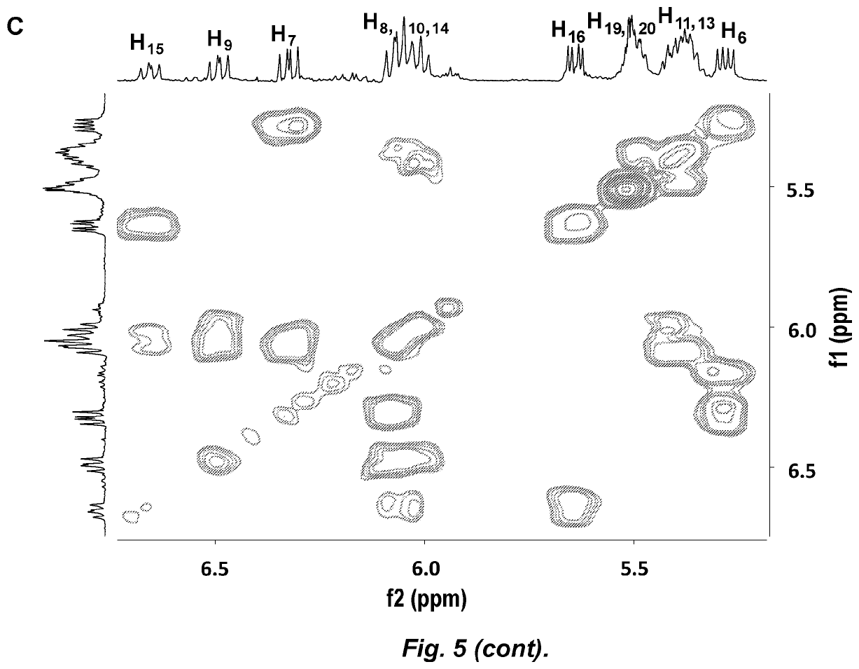

FIGS. 5A, 5B and 5C provide NMR validation of 4,5 epoxy-resolvin. 5A) UV chromophore for 4S, 5S-epoxy-17S-hydroxy-resolvin methyl ester with λₘₐₓ^{MeOH}=281 nm for the conjugated triene and 228 nm for the diene moieties, respectively. 5B) Structure of 4S, 5S-epoxy-17S-hydroxy-resolvin methyl ester denoting hydrogens detected by two-dimensional COSY NMR. 5C) Two-dimensional COSY NMR of 4S, 5S-epoxy-17S-hydroxy-resolvin methyl ester. Double-bond geometry was assigned using 2D ¹H-¹H NMR on a Varian NMRS 600 MHz NMR spectrometer at 25° C. on a 5 mm Triple Resonance PFG¹H probe and referenced to the benzene-d₆ (C₆D₆) internal standard. The rainbow plot depicts positive contours of cross peaks along the diagonal axis allowing for the full proton assignment. The zoomed-in region highlights the Z/E olefinic protons H₆-H₁₁, H₁₃-H₁₆ and H₁₉-H₂₀.

### Example 9

### Flow Cytometry

Human neutrophils were enumerated and assessed for viability by Trypan blue followed by fixation with a 4% paraformaldehyde solution. FcR-mediated, nonspecific antibody binding was blocked with Human TruStain FcX solution (BioLegend). Human cells were extracellularly stained with anti-human PerCP-Cy5.5-conjugated CD66b (G10F5, Biolegend). Primary antibody staining was first optimized and occurred individually in 1X FoxP3 Perm Buffer (Biolegend) at the following dilutions: 1:500 LTC₄S (polyclonal, Abcam, Cambridge, MA, USA), 1:500 mGST2 (polyclonal, Abcam), 1:500 mGST3 (polyclonal, Abcam), or 1:1000 GSTM4 (polyclonal, Thermo Fisher Scientific). Secondary antibody staining occurred in 1X FoxP3 Perm Buffer with 1:250 F(ab′)2-donkey anti-rabbit PE-conjugated IgG H+L (polyclonal, Thermo Fisher Scientific). Samples were analyzed with a FACSCanto Flow Cytometer (BD Biosciences, San Jose, CA, USA) and FlowJo 10 software (FlowJo LLC, Ashland, OR USA). Debris (FSC-A vs SSC-A) and doublets (FSC-H vs FSC-A FSC-A vs FSC-W SSC-H vs SSC-A) were excluded before further gating on populations of interest. Unstained controls were used for differentiating cell versus antibody fluorescence as well as single stain controls for calculating fluorochrome wavelength overlap compensation. Data is represented as mean fluorescence intensity (MFI) of individual enzyme expression in CD66b⁺ cells.

### Example 10

Human Neutrophils (PMN) Biosynthesize RvD3 and RvD4 from 4S, 5S-epoxy-Resolvin

Human neutrophils (1×10⁶ cells/mL) were incubated with either leukotriene A₄ (500 ng) (obtained by hydrolysis of LTA₄ methyl ester with lithium hydroxide as described in Chang 1987 Arch Biochem Biophys) or 4S, 5S-epoxy-resolvin (500 ng) for 15 min at 37° C. in PBS⁺⁺ (pH 7.4). Ice cold methanol was added, and incubations were taken to LC-MS/MS analysis.

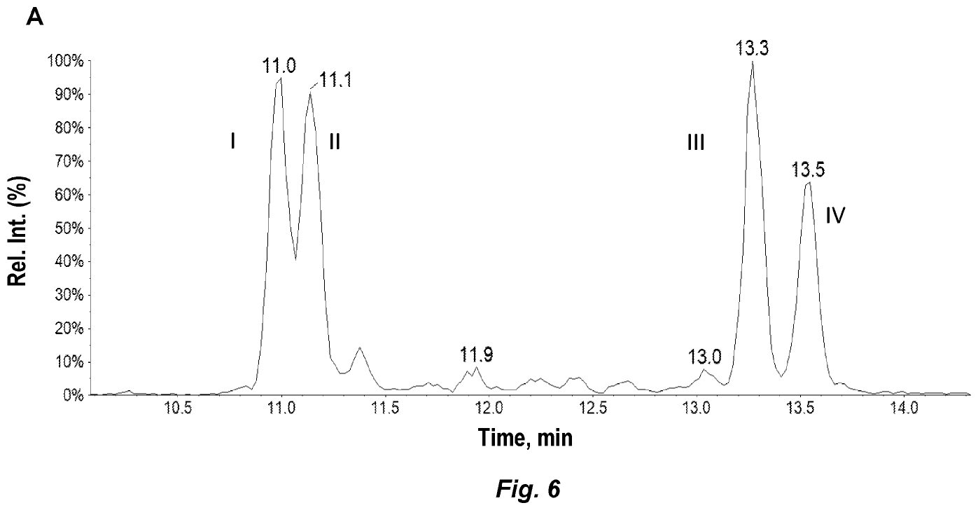

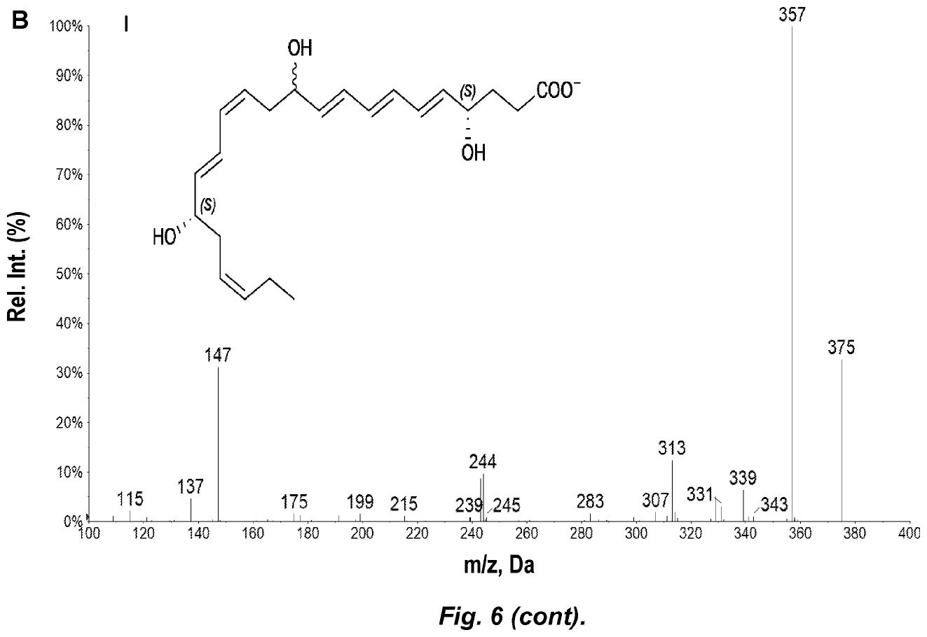

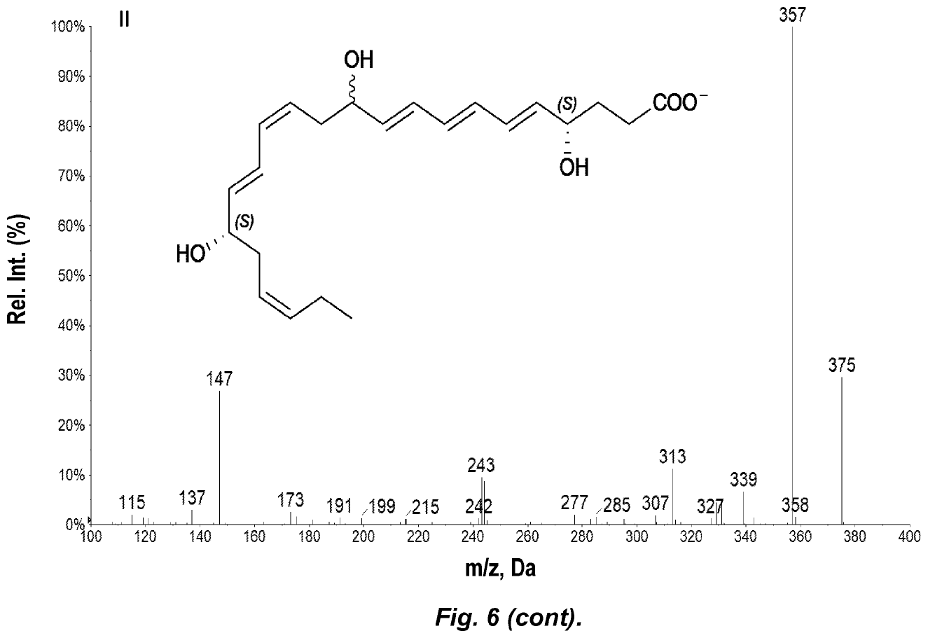

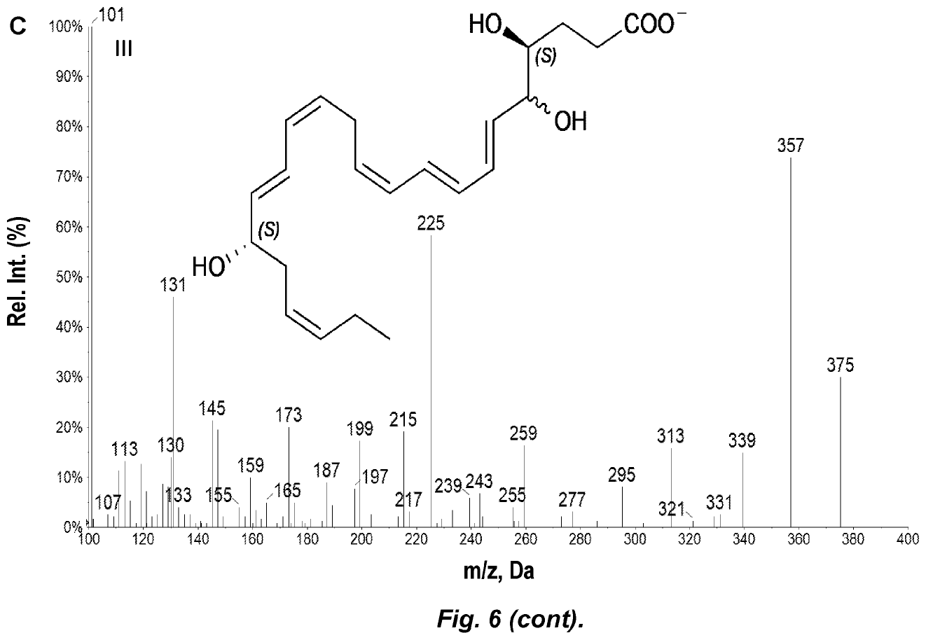

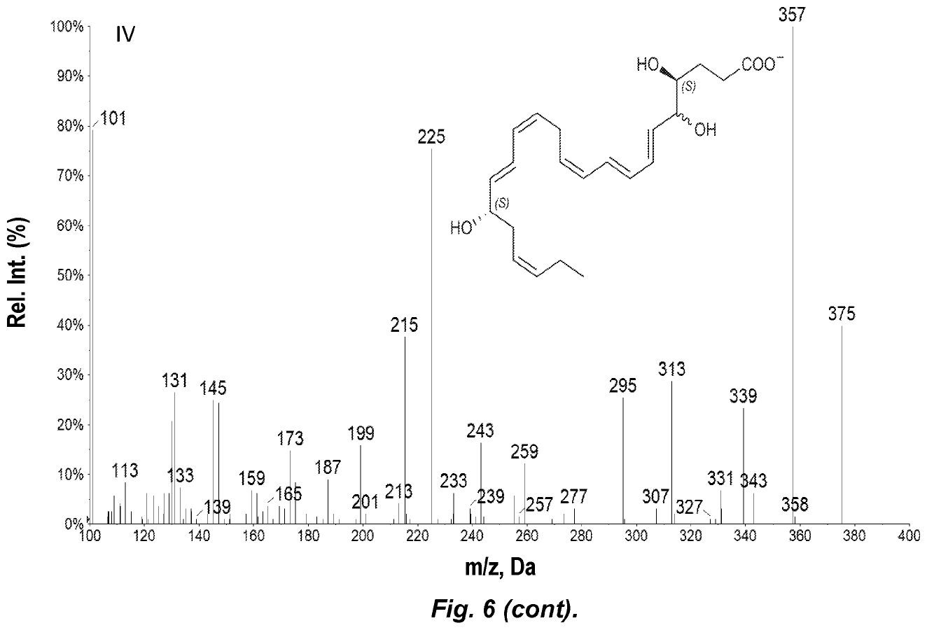

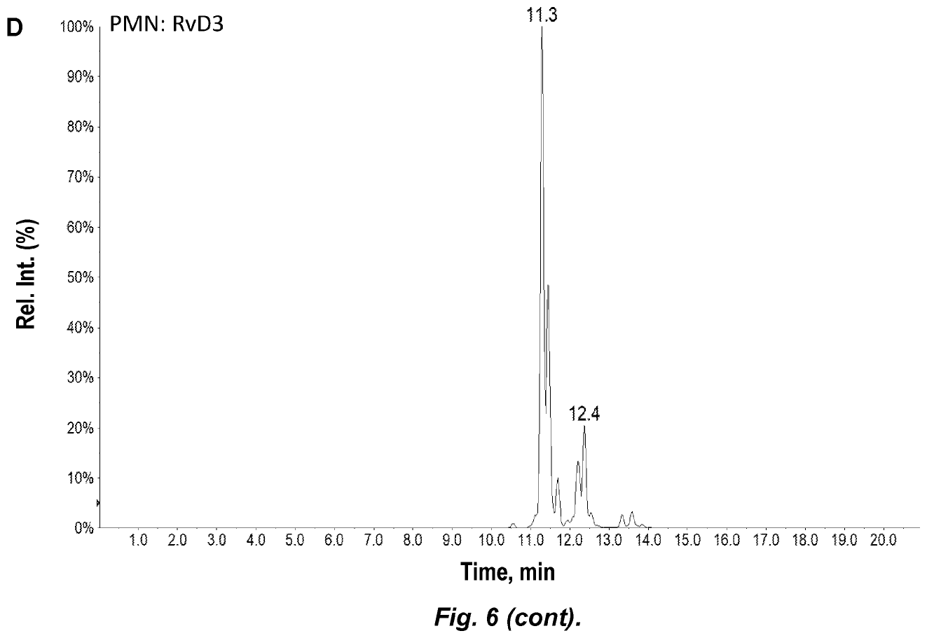

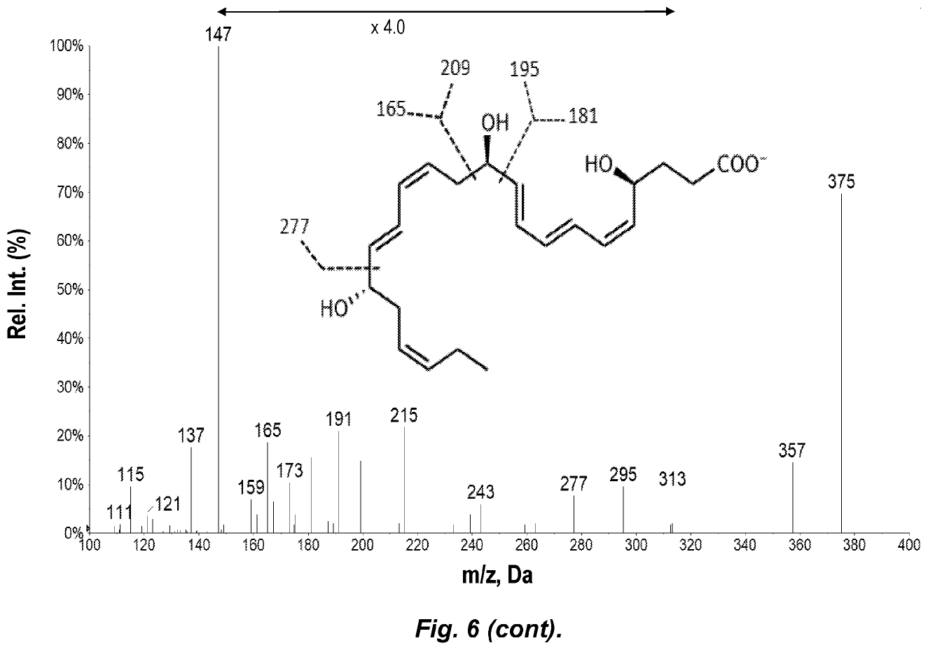

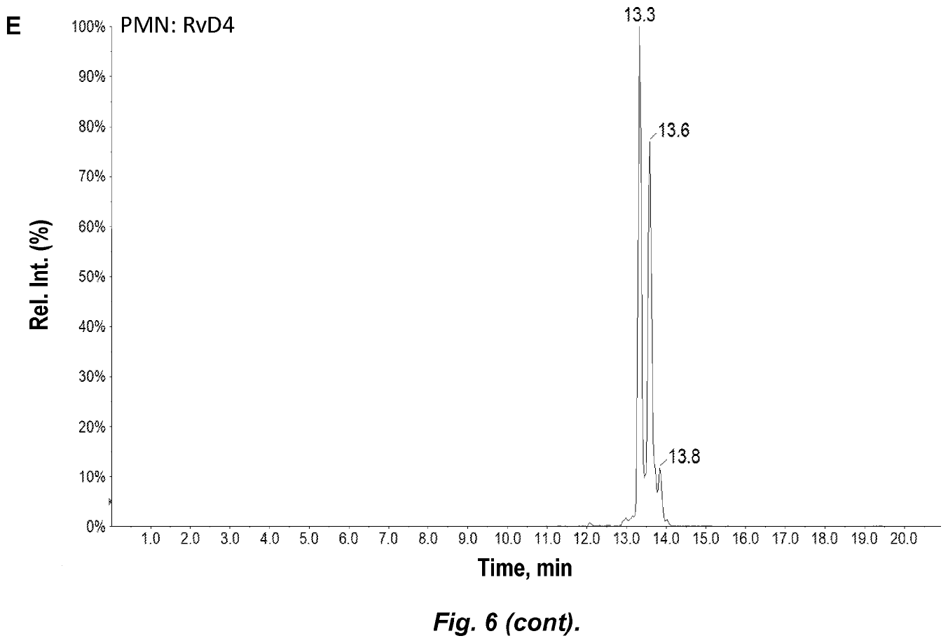

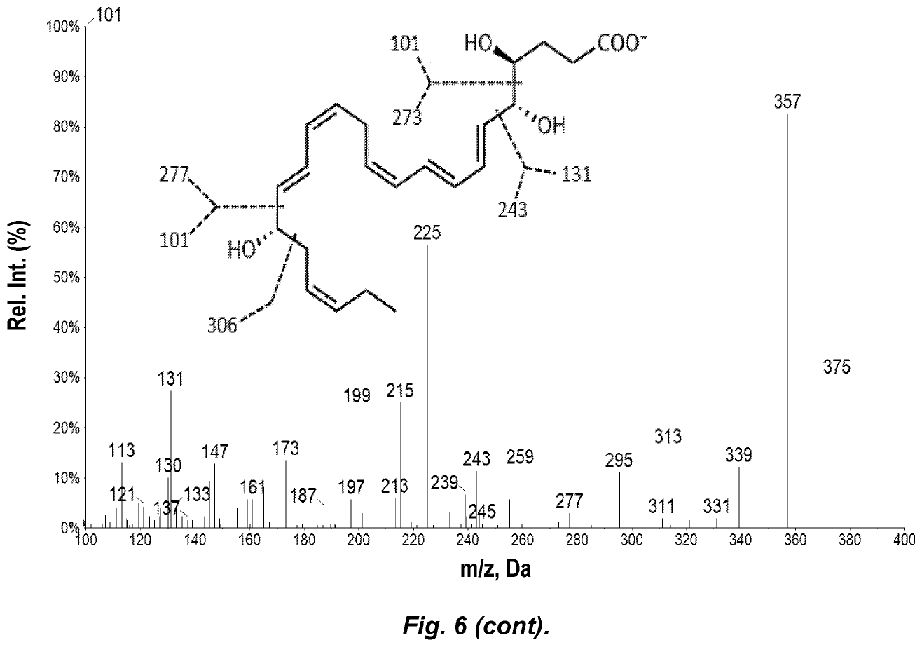

The 4S, 5S-epoxy-resolvin (1 μg) was incubated for 5 min at 37° C. in PBS⁺⁺ (pH 7.4) to obtain the aqueous hydrolysis profile for direct comparisons shown in FIGS. 6A, 6B,6C, 6D and 6E. 6A) LC-MS/MS MRM monitoring in negative ion mode for m/z 375>147. 6B) Structure and MS/MS fragmentation spectrum with proposed ions matching the non-enzymatic hydrolysis products I (left) and II (right). 6C) Structure and MS/MS fragmentation spectrum with proposed ions matching the non-enzymatic hydrolysis products III (left) and IV (right). 6D) Human neutrophils (1×10⁶) were incubated with 4S, 5S-epoxy-resolvin (500 ng) for 15 min at 37° C. in 500 μL PBS⁺⁺ (pH 7.4). (left) LC-MS/MS MRM monitoring in negative ion mode for m/z 375>147 (RvD3). (right) Structure and MS/MS fragmentation spectrum with proposed ions matching RvD3. 6E) (left) LC-MS/MS MRM monitoring in negative ion mode for m/z 375>101 (RvD4) and structure. (right) MS/MS fragmentation spectrum with proposed ions matching RvD4.

### Example 11

Human Neutrophils Express Glutathione S-Transferases and Biosynthesize Novel Peptido-Lipid Mediator from 4S, 5S-epoxy-Resolvin

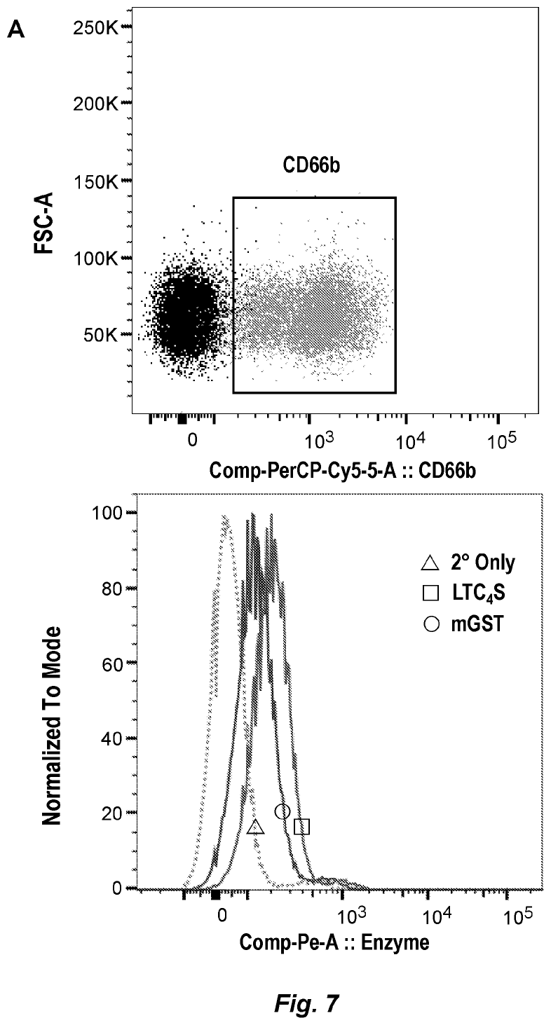

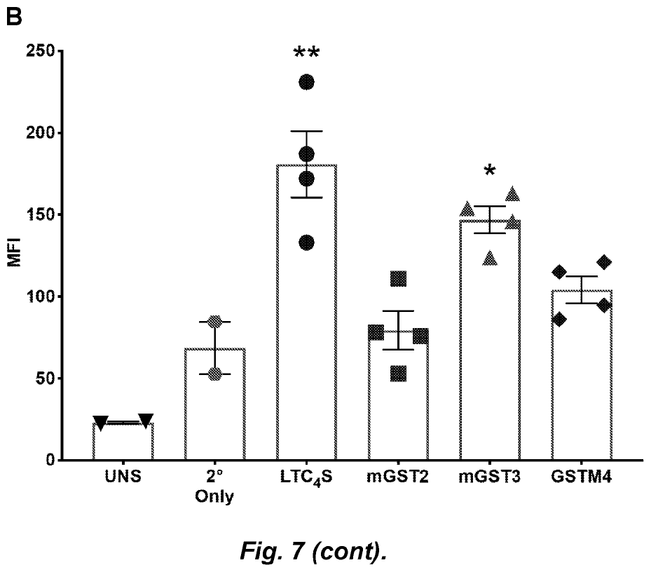

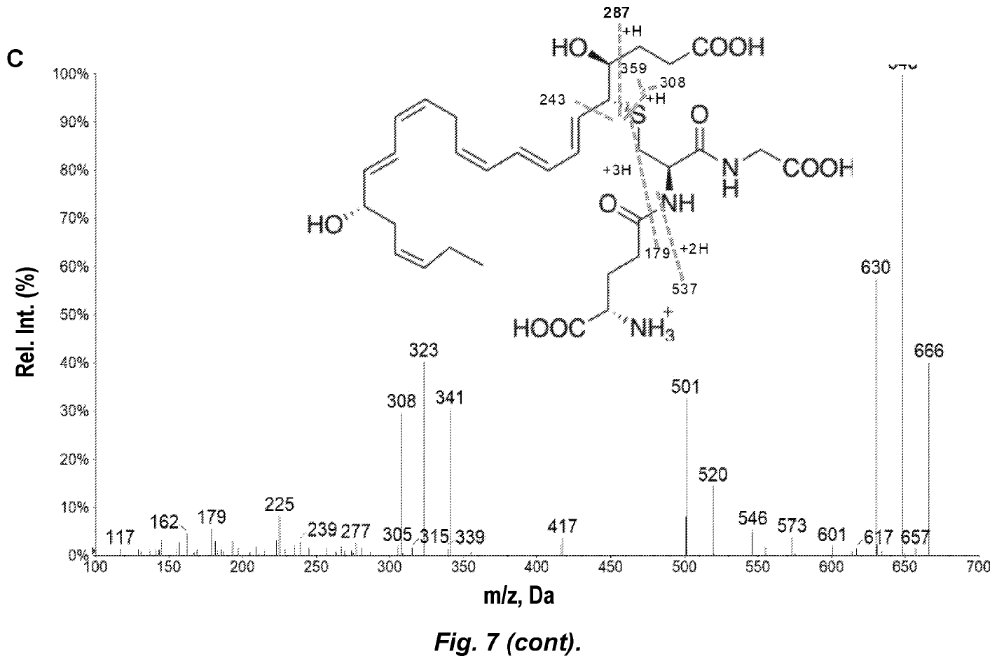

FIGS. 7A, 7B, and 7C. Human neutrophils were isolated from peripheral blood of donors and subjected to flow cytometry. Debris and doublets were first gated out before further gating on CD66b followed by LTC₄S, mGST2, mGST3, and GSTM4. 7A) (left) Representative gating depicting the unstained cell population (black) and CD66b⁺ (blue). (right) Representative histogram of LTC₄S and mGST3 expression. 7B) Mean fluorescent intensity (MFI) of LTC₄S, mGST2, mGST3, and GSTM4 in CD66b⁺ cells. n=4 individual human donors. Mean±SEM. Ordinary One-way ANOVA with Tukey multiple comparisons test. *p<0.05 and **p<0.01 versus secondary only. 7C) Human neutrophils (1×10⁶) were incubated with 4S, 5S-epoxy-resolvin (500 ng) for 15 min at 37° C. in 500 μL PBS⁺⁺ (pH 7.4). Structure and MS/MS fragmentation spectrum with proposed ions matching Product 1.

### Example 12

Human Neutrophils Biosynthesize Cysteinyl-Leukotrienes from LTA₄

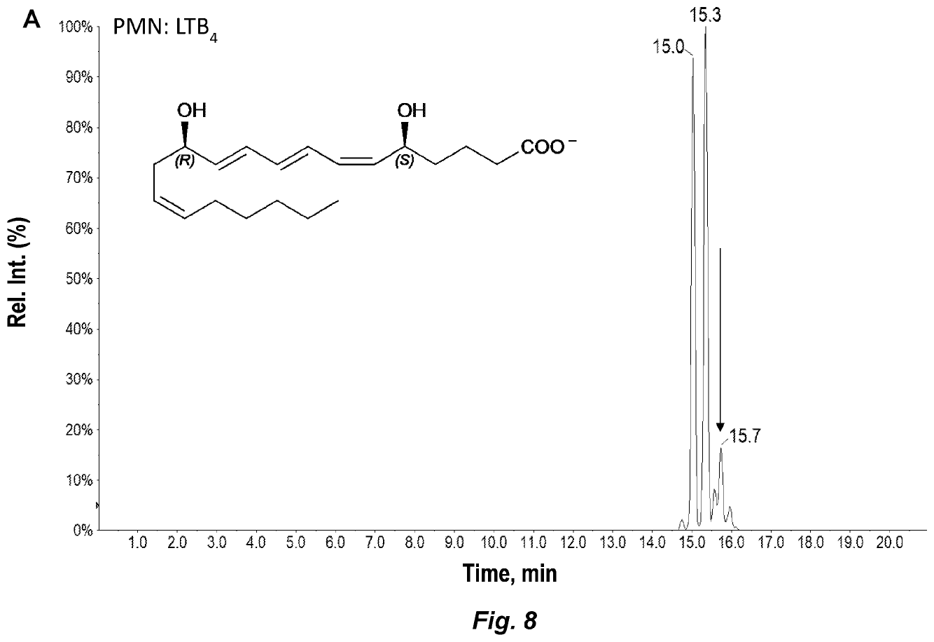

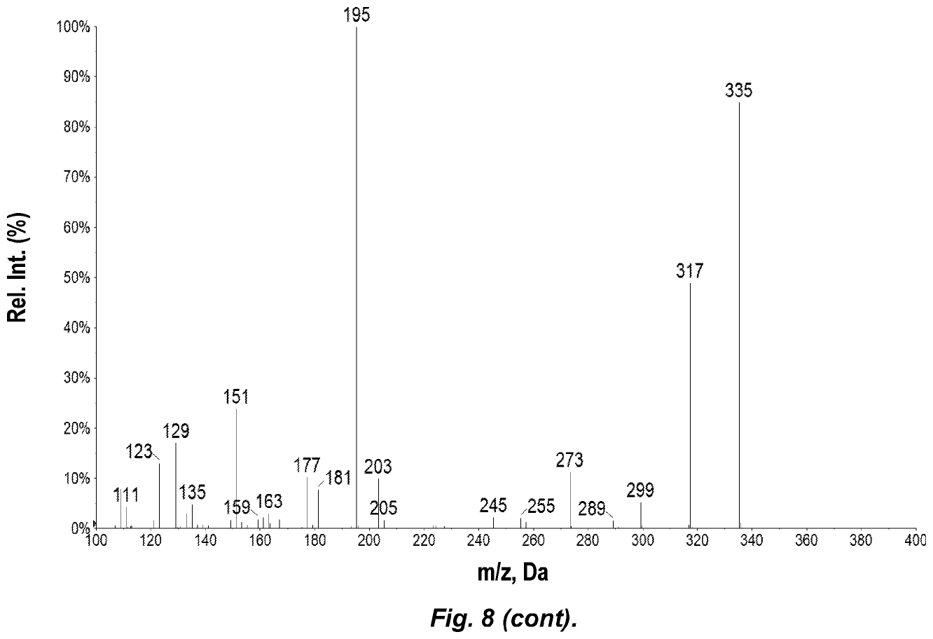

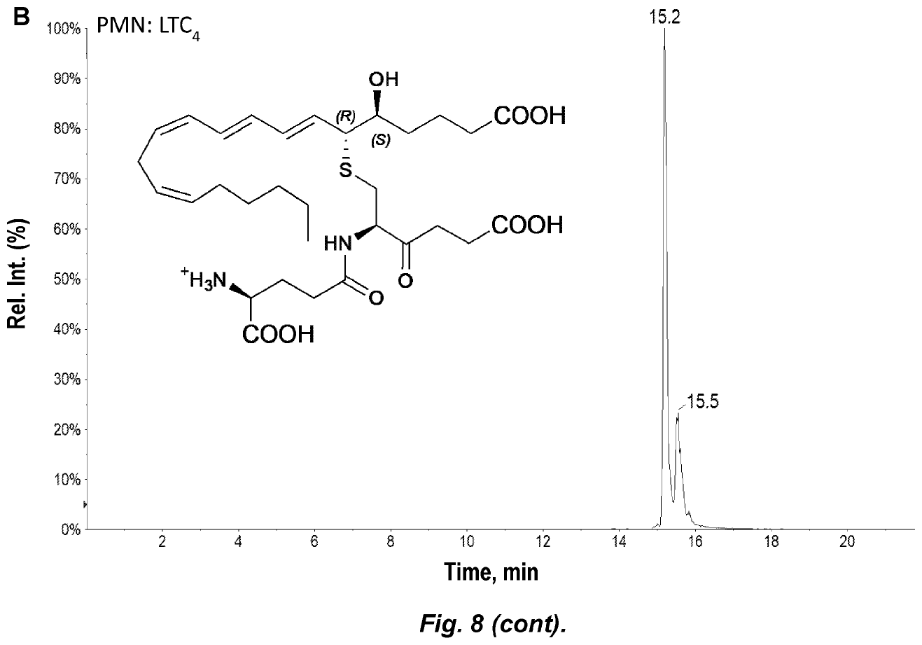

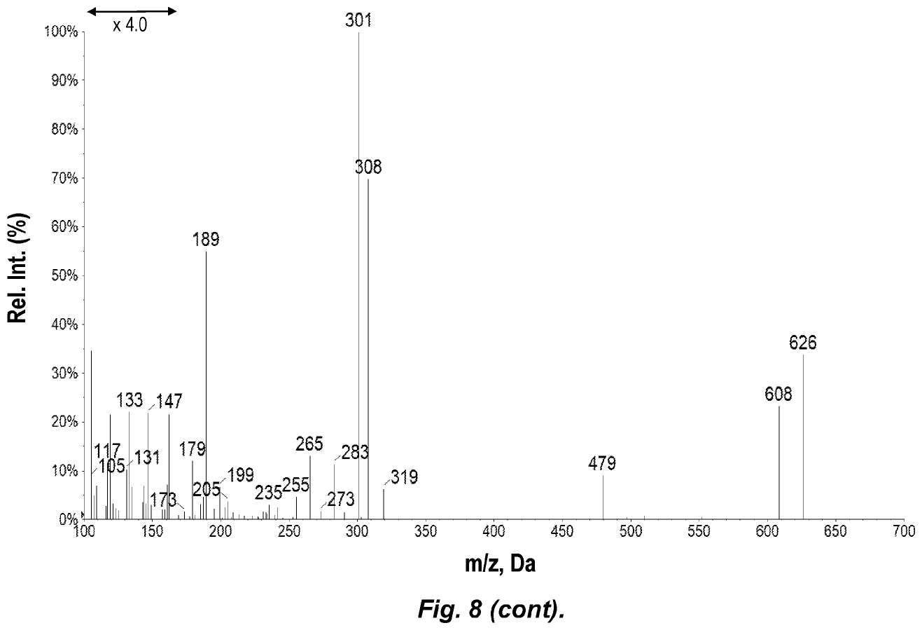

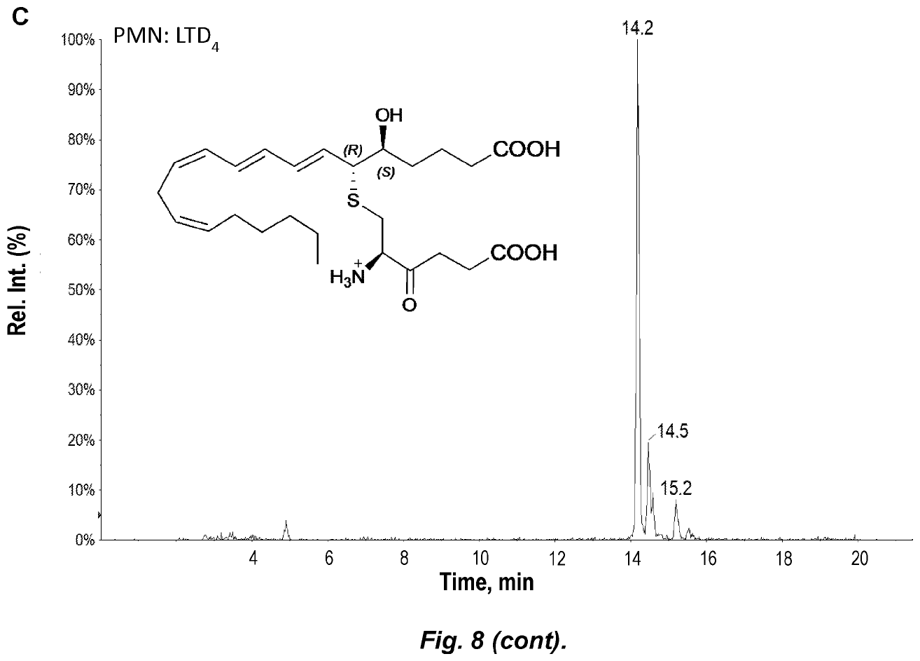

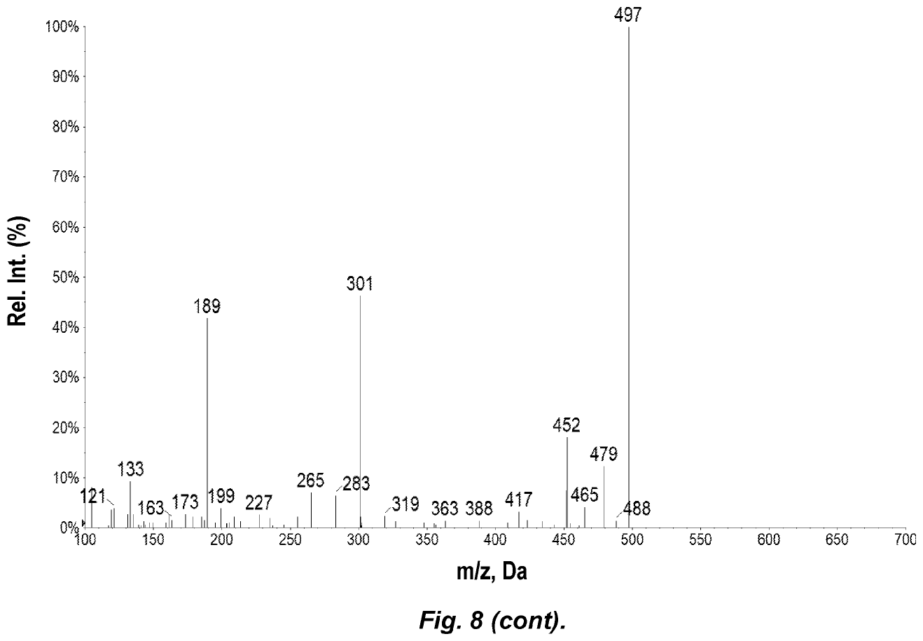

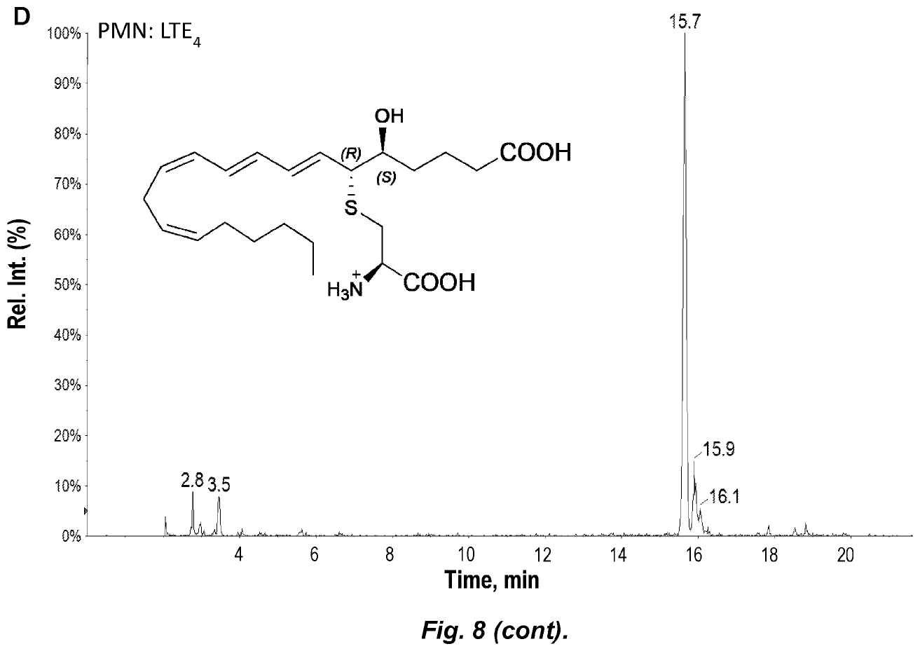

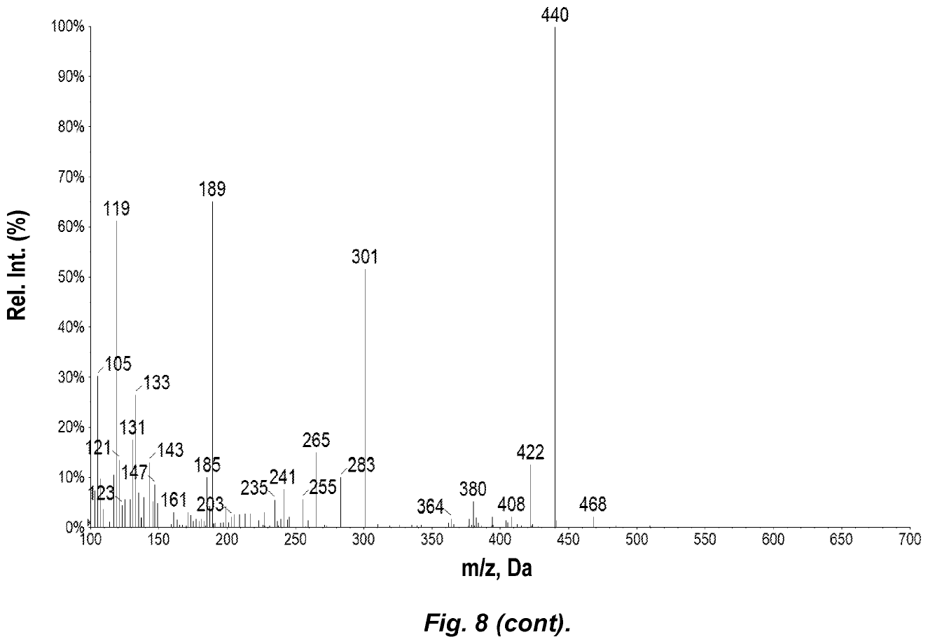

FIGS. 8A, 8B, 8C and 8D. Human neutrophils (1×10⁶) were incubated with leukotriene A₄ (500 ng) for 15 min at 37° C. in 500 μL PBS⁺⁺ (pH 7.4). 8A) (left) Structure and LC-MS/MS MRM monitoring in negative ion mode for m/z 335>195 (LTB₄) (right) MS/MS fragmentation spectrum with proposed ions matching LTB₄. 8B) (left) Structure and LC-MS/MS MRM monitoring in positive ion mode for m/z 626>189 (LTC₄) (right) MS/MS fragmentation spectrum with proposed ions matching LTC₄. 8C) (left) Structure and LC-MS/MS MRM monitoring in positive ion mode for m/z 497>189 (LTD₄) (right) MS/MS fragmentation spectrum with proposed ions matching LTD₄. 8D) (left) Structure and LC-MS/MS MRM monitoring in positive ion mode for m/z 440>189 (LTE₄) (right) MS/MS fragmentation spectrum with proposed ions matching LTE₄.

### Example 13

### Macrophages

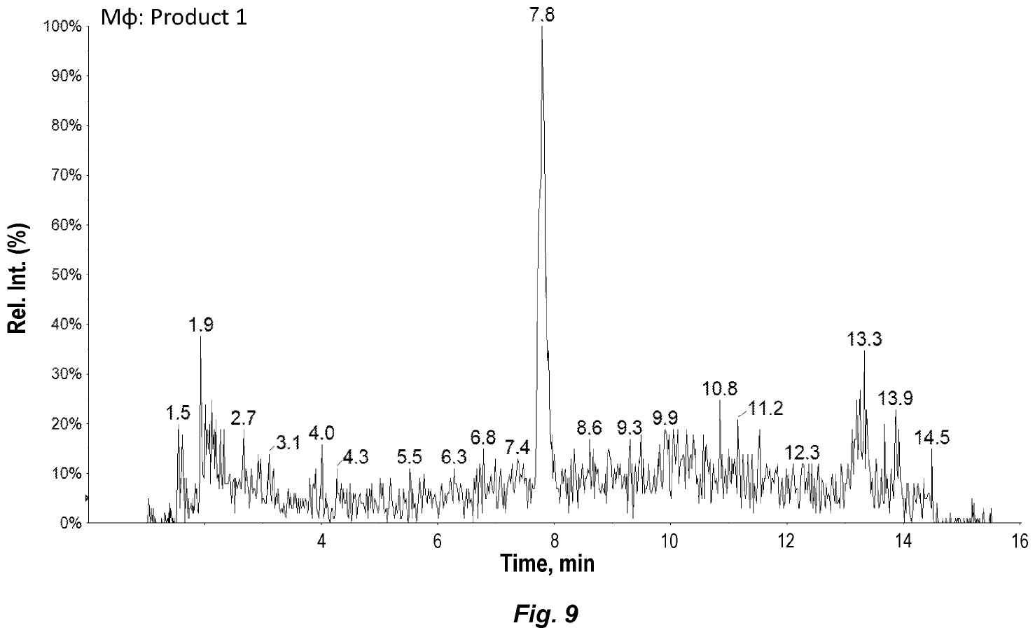

Human macrophages (10⁷ cells/mL) were incubated with 4S, 5S-epoxy-resolvin (1 1 μg) for 5 min at 37° C. in PBS⁺⁺ (pH 7.4). Ice cold methanol was added, and incubations were taken to LC-MS/MS analysis. FIG. 9 LC-MS/MS MRM monitoring in positive ion mode for m/z 666>648 (Product 1).

### Example 14

Human Macrophages Biosynthesize Novel Peptido-Lipid Mediator (Product 2) from 4S, 5S-epoxy-Resolvin

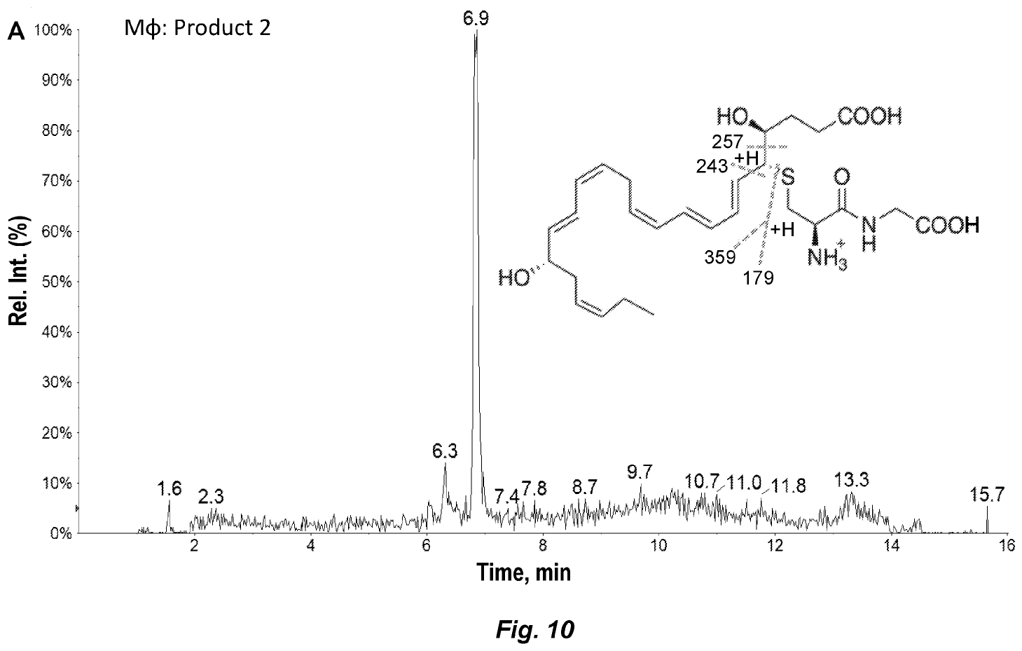

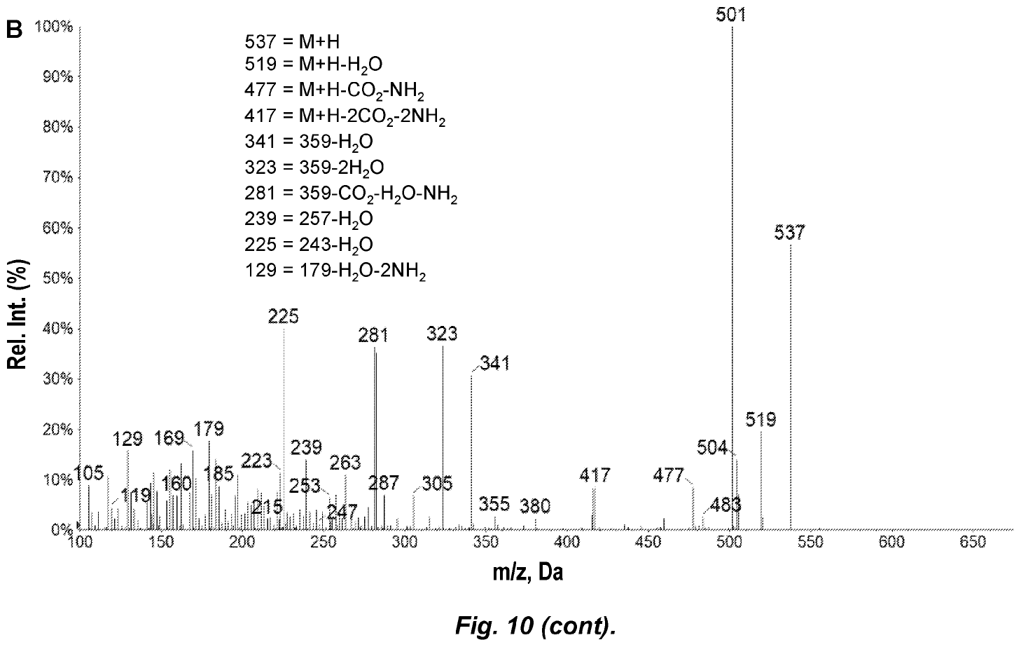

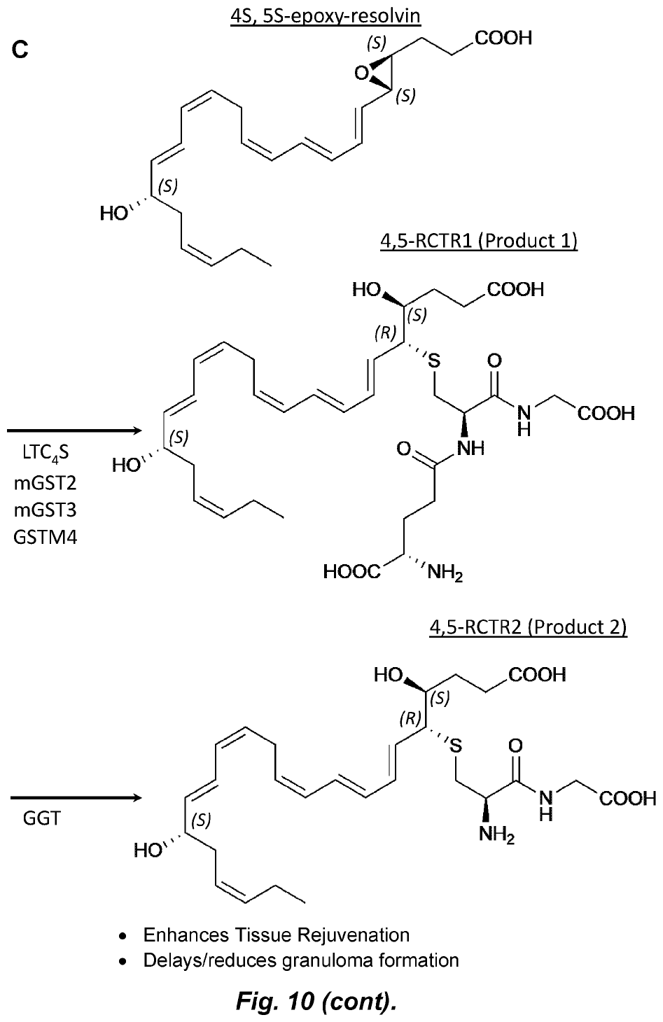

FIGS. 10A, 10B and 10C. Human macrophages (10⁷ cells/mL) were incubated with 4S, 5S-epoxy-resolvin (1 μg) for 5 min at 37° C. in PBS⁺⁺ (pH 7.4). 10A) LC-MS/MS MRM monitoring in positive ion mode for m/z 537>501 (Product 2) and theoretical structure. 10B) MS/MS fragmentation spectrum with proposed ions matching Product 2. 10C) Proposed biosynthetic pathway of Product 1 and Product 2.

### Example 15

Structural Similarity of Novel Lipid 4S,5S-epoxy-resolvin Peptido-Lipids to RCTR1 and RCTR2

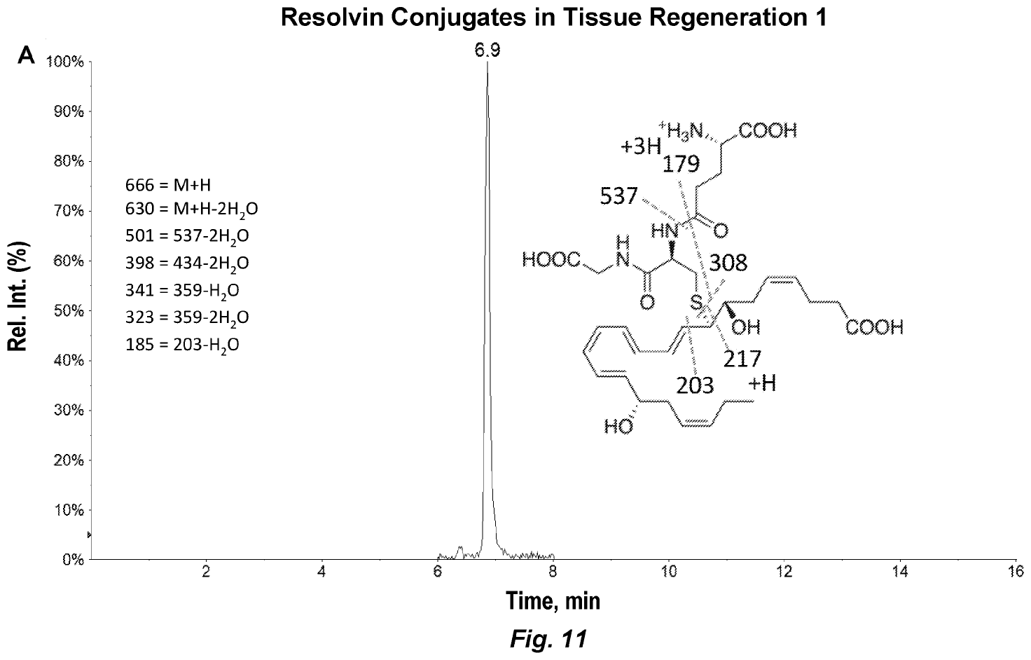

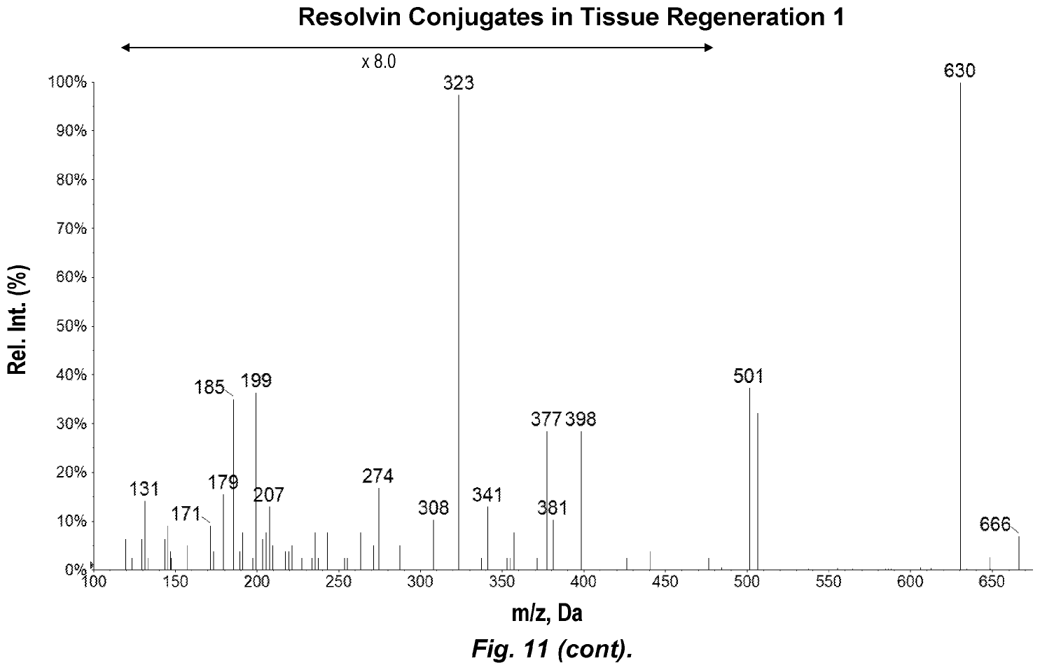

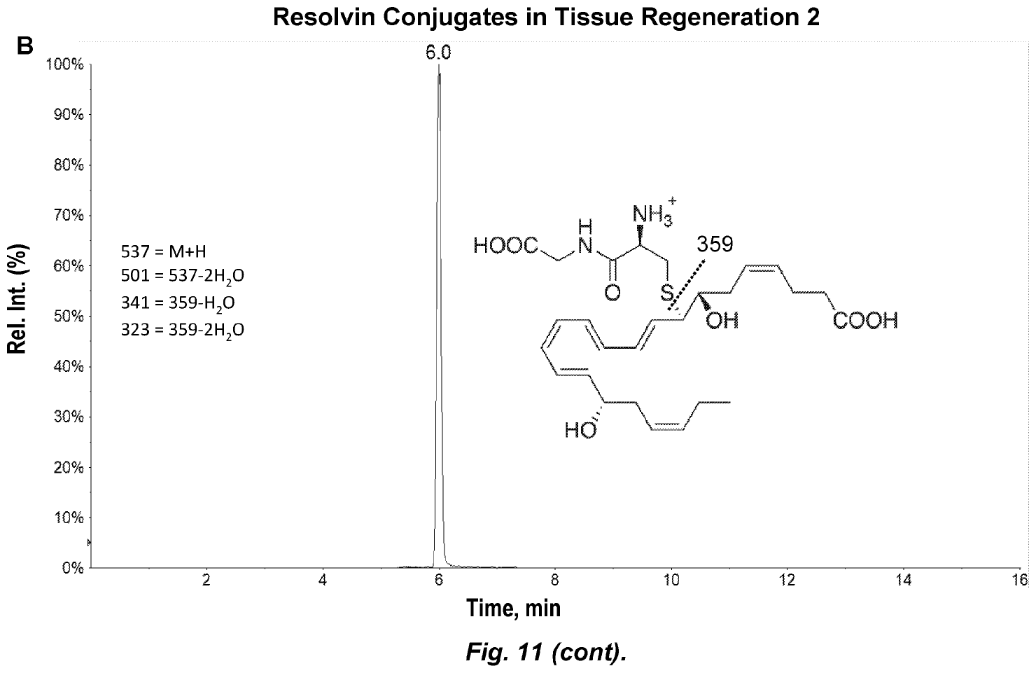

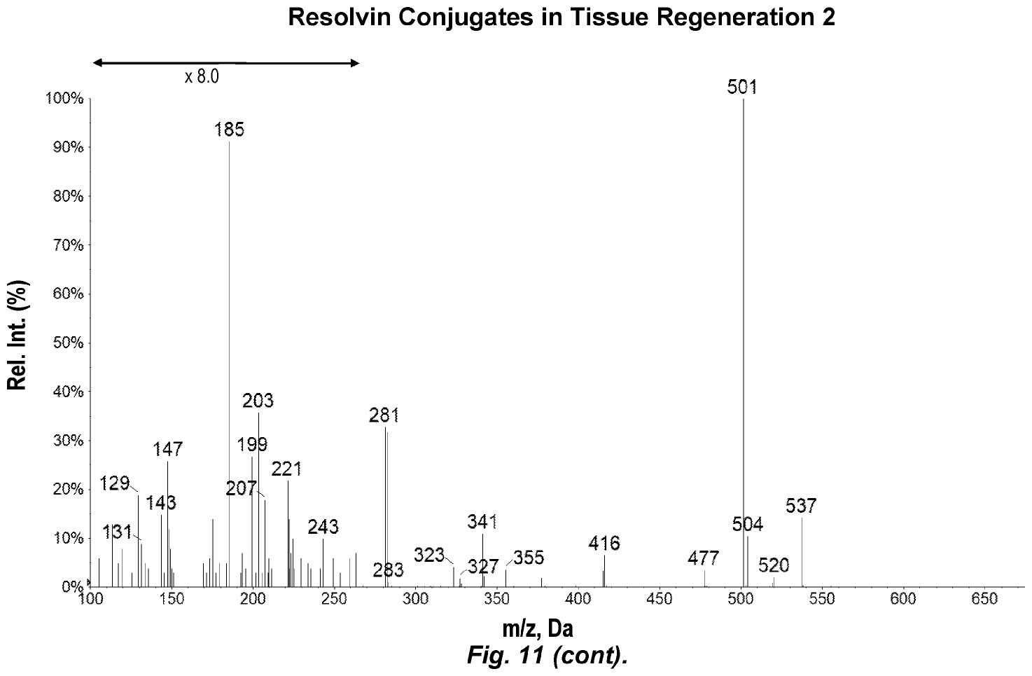

FIGS. 11A, 11B, 11C and 11D. Authentic total organic synthesized standards for RCTR1 and RCTR2. 11A) LC-MS/MS MRM monitoring in positive ion mode for m/z 666>630 (RCTR1) and structure. 11B) MS/MS fragmentation spectrum with proposed ions matching RCTR1. 11C) LC-MS/MS MRM monitoring in positive ion mode for m/z 537>501 (RCTR2) and structure. 11D) MS/MS fragmentation spectrum with proposed ions matching RCTR2.

### Example 16

Recombinant Human Enzymatic Conjugation of 4S, 5S-epoxy-resolvin to Product 1

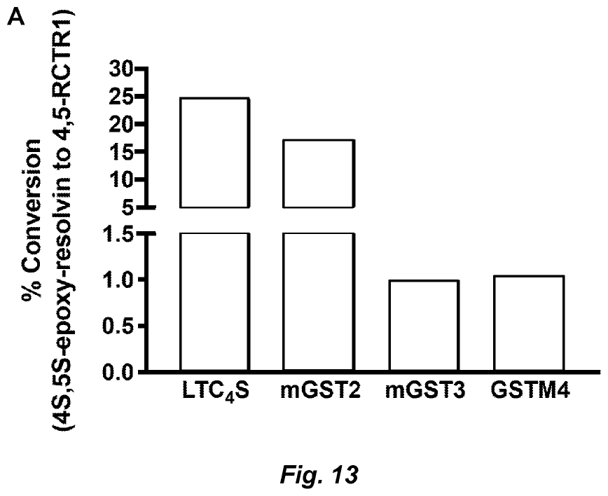

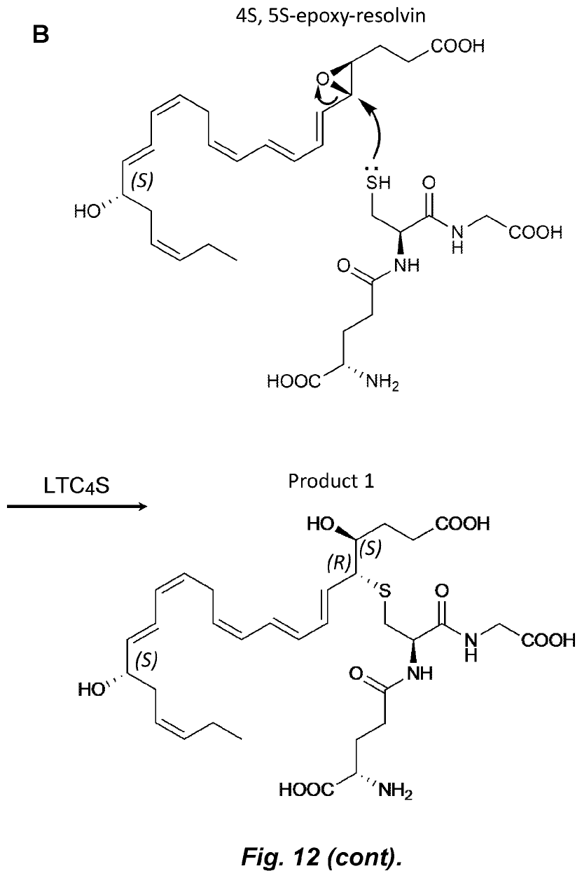

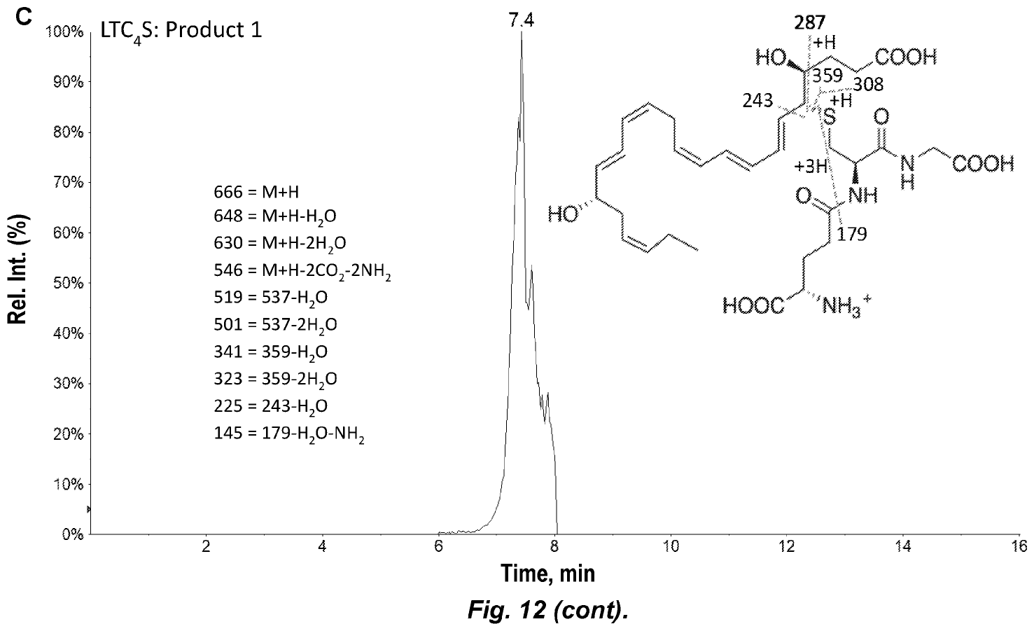

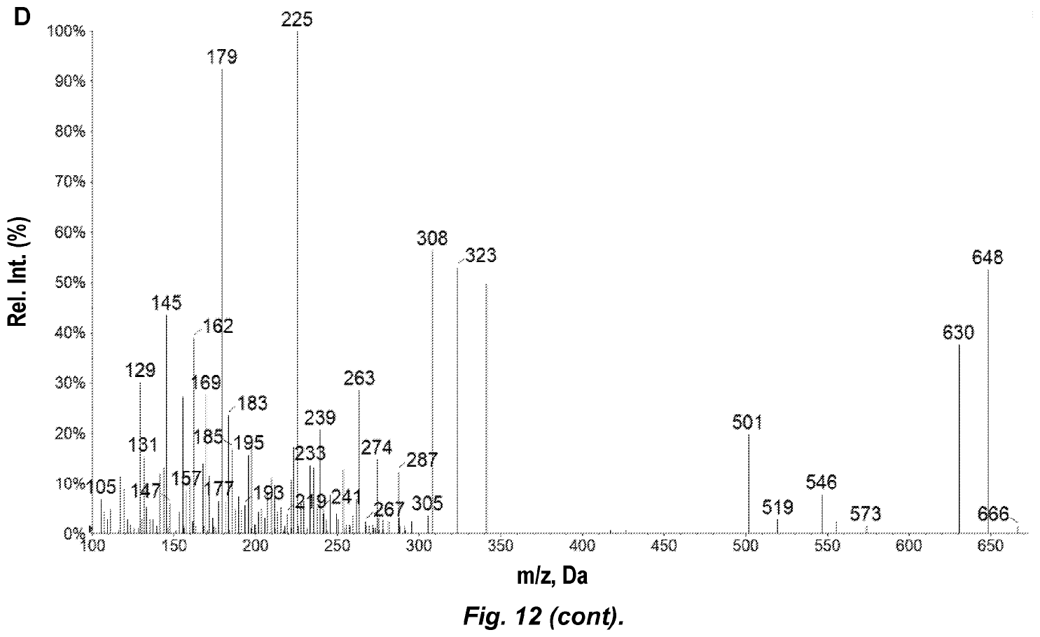

FIGS. 12A, 12B, 12C and 12D. Human recombinant LTC₄S (2 μg), mGST2 (2 μg), mGST3 (2 μg), and GSTM4 (2 μg) were incubated with 4S, 5S-epoxy-resolvin (100 ng) and reduced glutathione (5 mM) in 25 mM Tris-HCl (pH 8.0), 0.05% Triton X-100 for 15 min at 37° C. 12A) The percent conversion of 4S, 5S-epoxy-resolvin to Product 1 by LTC₄S, mGST2, mGST3, and GSTM4. 12B) Scheme of SN2 reaction of reduced glutathione and 4S, catalyzed by LTC₄S. 12C) LC-MS/MS MRM monitoring in positive ion mode for m/z 666>648 (Product 1) and structure. 12D) MS/MS fragmentation spectrum with proposed ions matching Product 1.

### Example 17

### Liquid Chromatography Tandem Mass Spectrometry

Ice-cold methanol containing deuterium labeled internal standards including d5-RvD3, d5-LTC₄, d5-LTD₄, and (¹³C)₂¹⁵ N-MCTR3 (500 μg each) were added to each sample before lipid extraction for quantification and recovery of the lipid mediators (LM). After centrifugation at 1000 g for 10 min at 4° C., supernatants were collected, and LM were extracted per optimized methods using an automated extractor (Extrahera, Biotage, Charlotte, NC, USA) as described in (De La Rosa 2018 AJP, Norris 2018 Sci Rep, Jouvene 2019 FASEB J). Samples were acidified to an apparent pH 3.5, loaded onto 3 mL-SPE Isolute C18 100 mg cartridges (Biotage, Charlotte, NC, USA), and rapidly neutralized with double-distilled water. Methyl formate was used to elute RvD3, RvD4, LTB₄, and non-enzymatic hydrolysis products and methanol was used to elute LTC₄, LTD₄, LTE₄, RCTRs, and 4,5-RCTRs. Both fractions were brought to dryness under a gentle stream of nitrogen gas using an automated evaporation system (TurboVap LV, Biotage), and immediately resuspended in a methanol-water mixture (50:50, v/v) for LC-MS/MS analysis. Samples were acquired on either a LC-MS/MS consisting of a 5500 QTRAP (Sciex, Framingham, MA, USA) equipped with a LC-20AD UFLC (Shimadzu, Tokyo, Japan). A Poroshell 120 EC-C18 column (100 mm×4.6 mm×2.7 μm; Agilent Technologies, Santa Clara, CA, USA) was kept in a column cover regulated at 50° C. Or a LC-MS/MS consisting of a 6500⁺ QTRAP in low mass mode (Sciex) equipped with an ExionLC (Shimadzu). A Kinetex Polar C18 (100 mm×4.6 mm×2.6 μm; Phenomenex, Torrance, CA, USA) was kept in a column cover regulated at 50° C. Please refer to Tables 1 and 2 which specifies instrumentation as well as Q1 (m/z), Q3 (m/z), declustering potential (DP), entrance potential (EP), collision energy (CE) and collision cell exit potential (CXP) for each mediator. The solvent gradients and instrumentation settings are described in (Table 3). To monitor and quantify RvD3, RvD4, LTB₄, and non-enzymatic hydrolysis products, targeted multiple reaction monitoring (MRM) (Table 1) and enhanced product ion (EPI) in a negative mode (ion spray voltage: −4200 V) were used. To monitor and quantify LTC₄, LTD₄, LTE₄, RCTRs, and 4,5-RCTRs, targeted MRM (Table 2) and EPI in a positive mode (ion spray voltage: 5200 V) were used. Limit of detection is ˜0.1 μg. For each known compound, linear calibration curves were obtained using synthetic authentic standards with r 2 values of 0.98 to 0.99. Reported criteria were used for identification of each molecule, including MS/MS matching to at least six diagnostic ion fragments per molecule, and a matching retention time to authentic synthetic standards, where the synthetic standards were also qualified by NMR. LC-MS/MS MRM trace data and EPI spectral data are presented as screen captures from Analyst 1.6.2 and 1.7 (Sciex).

TABLE 1
Mass Spectrometer Settings in Negative Mode for Multiple Reaction
Monitoring of RvD3, RvD4, LTB₄, and Hydrolysis Products I-IV
on a Sciex 5500 and 6500⁺ Low Mass QTRAP. This table lists the
lipid mediator of interest's name, mass spectrometer, Q1 (m/z),
Q3 (m/z), declustering potential (DP, V), entrance potential
(EP, V), collision energy (CE, V), and collision cell exit
potential (CXP, V).

| | | Q1 | Q3 | DP | EP | CE | CXP |
|---|---|---|---|---|---|---|---|
| Name | MS | (m/z) | (m/z) | (V) | (V) | (V) | (V) |
| RvD3 | 5500/ | 375 | 147 | −80/ | −10 | −25 | −13/ |
| | 6500⁺ | | | −40 | | | −12 |
| RvD4 | 5500/ | 375 | 101 | −80/ | −10 | −22 | −16/ |
| | 6500⁺ | | | −40 | | | −12 |
| 4,5-eRv Hydrolysis | 6500⁺ | 375 | 147 | −40 | −10 | −25 | −12 |
| Products I and II | | | | | | | |
| 4,5-eRv Hydrolysis | 6500⁺ | 375 | 147 | −40 | −10 | −25 | −12 |
| Products III and IV | | | | | | | |
| LTA₄ Hydrolysis | 6500⁺ | 335 | 195 | −40 | −10 | −22 | −12 |
| Products I and II | | | | | | | |
| LTA₄ Hydrolysis | 6500⁺ | 335 | 115 | −40 | −10 | −22 | −12 |
| Products III and IV | | | | | | | |
| LTB₄ | 6500⁺ | 335 | 195 | −40 | −10 | −22 | −12 |

TABLE 2
Mass Spectrometer Settings in Positive Mode for Multiple Reaction
Monitoring of Peptido-Lipid Mediators on a Sciex 5500 and 6500⁺
Low Mass QTRAP. This table lists the lipid mediator of interest's
name, mass spectrometer, Q1 (m/z), Q3 (m/z), declustering potential
(DP, V), entrance potential (EP, V), collision energy (CE, V),
and collision cell exit potential (CXP, V).

| | | Q1 | Q3 | DP | EP | CE | CXP |
|---|---|---|---|---|---|---|---|
| Name | MS | (m/z) | (m/z) | (V) | (V) | (V) | (V) |
| RCTR1 | 5500 | 666 | 323 | 80 | 13 | 24 | 13 |
| RCTR2 | 5500 | 537 | 501 | 80 | 10 | 25 | 13 |
| 4,5-RCTR1 | 5500 | 666 | 225 | 80 | 10 | 25 | 13 |
| 4,5-RCTR2 | 5500 | 537 | 225 | 80 | 10 | 20 | 13 |
| LTC₄ | 6500⁺ | 626 | 189 | 40 | 10 | 28 | 13 |
| LTD₄ | 6500⁺ | 497 | 189 | 40 | 10 | 23 | 13 |
| LTE₄ | 6500⁺ | 440 | 189 | 40 | 10 | 23 | 13 |

TABLE 3
Shimadzu LC20AD and ExionLC and Sciex 5500 and 6500⁺ Low mass
QTRAP Settings. This table lists the following information for the LC20AD,
ExionLC, Sciex 5500 (polarity: negative), and 6500⁺ QTRAP low
mass (polarity: negative and positive): columns, solvents, flow rates,
gradients, curtain gas, collision gas, ion spray voltage, temperature,
ion source gas 1, and ion source gas 2 for multiple reaction monitoring
(MRM) and enhanced production (EPI) modes.

| | Shimadzu LC20AD | | Shimadzu ExionLC | |
|---|---|---|---|---|
| Column | Poroshell 120 EC-C18 | | Kinetex Polar C18 | |
| | (100 mm × 4.6 mm × 2.7 μm) | | (100 mm × 4.6 mm × 2.6 μm) | |
| | Agilent | | Phenomenex | |
| Solvent A | Water (0.1% acetic acid) | | Water (0.1% formic acid) | |
| Solvent B | Methanol (0.1% acetic acid) | | Methanol (0.1% formic acid) | |
| Flow Rate | 0.6 mL/min | | 0.5 mL/min | |
| | Time (min) | Solvent B (%) | Time (min) | Solvent B (%) |
| Gradient | 1.0 | 45 | 0.1 | 45 |
| | 1.1 | 45 | 2.0 | 45 |
| | 6.0 | 30 | 16.5 | 80 |
| | 8.0 | 20 | 16.6 | 98 |
| | 11.0 | 20 | 18.5 | 98 |
| | 11.1 | 2 | 18.6 | 10 |
| | 14.0 | 2 | 20.5 | End |
| | 14.1 | 45 | — | — |
| | 16.0 | End | — | — |
| | | | Sclex 6500⁺ QTRAP | |
| | Sciex 5500 QTRAP | | Low Mass | |
| | MRM Mode | EPI Mode | MRM Mode | EPI MODE |
| Curtain Gas | 25.0 | 35.0 | 30.0 | 30.0 |
| Collison Gas | Medium | High | 12.0 | 12.0 |
| Ion Spray | 5200.0 | 5500.0 | −4200.0 | −4200.0 |
| Voltage | | | (+5200.0) | (+5200.0) |
| Temperature | 500.0 | 550.0 | 520.0 | 520.0 |
| Ion Source Gas 1 | 40.0 | 55.0 | 85.0 | 85.0 |
| Ion Source Gas 2 | 40.0 | 55.0 | 50.0 | 50.0 |

Multiple reaction monitoring (MRM) and enhanced product lon (EPI) modes

### Example 18

### Statistics

Statistical analyses were performed with GraphPad Prism software version 8 (GraphPad Software Inc., La Jolla, CA, USA), using unpaired two-tailed Student's t-tests or Ordinary One-Way ANOVA with Tukey multiple comparisons test. P values <0.05 were considered statistically significant.

### Example 19

### Novel Peptido-Lipid Mediator (4,5-RCTR1)

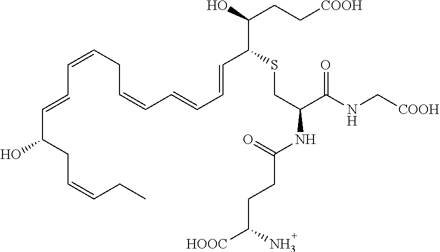 [SMILES: CC/C=C\C[C@H](O)/C=C/C=C\C\C=C/C=C/C=C/[C@@H](SC[C@H](NC(=O)CC[C@H](N)C(=O)O)C(=O)NCC(=O)O)[C@@H](O)CCC(=O)O]

Therapeutic human and veterinary use for 4,5-RCTR1 include but are not limited to:

Delaying or preventing granuloma formation in:
Mycobacterium Tuberculosis, Chronic Granulomatous Disease, Granulomatous steatitis, Foreign-body granulomas, Interstitial lung fibrosis (IPF), Leprosy, Arthritis, Sarcoidosis, Liver fibrosis, Heart fibrosis, Renal fibrosis, Hepatic cirrhosis, Pulmonary fibrosis, and Organ fibrosis.

Enhancing resolution of inflammatory diseases:
Oral inflammation, Periodontitis, Ulcerative colitis, Crohn's disease, Arthritis, Asthma and Chronic obstructive pulmonary disease, Hepatitis, Sinusitis, Systemic Lupus, Allergies, Dermatitis, Atherosclerosis, Psoriasis, Bronchitis, Appendicitis, Neurodegenerative diseases, and Multiple sclerosis,

Enhancing tissue regeneration.

### Example 20

### 3D Structure of 4,5-RCTR1

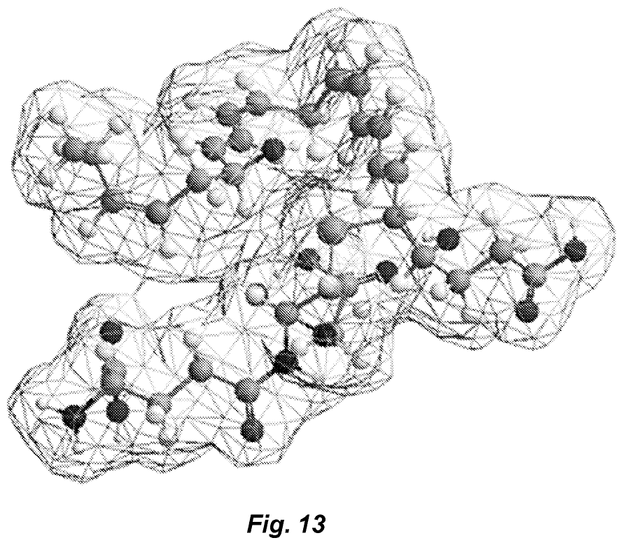

FIG. 13 discloses the 3-D structure of 4,5-RCTR1 confirmed by endogenous biosynthesis and spatial filling and was used to generate 4,5-RCTR1 mimetics/analogs disclosed in the following examples.

### Example 21

### Identification and Design of 4,5-RTCR1 Analogs

Because of the relatively sort half-life of 4,5-RTCR1 in the body, the design of analogs having a slower metabolism and longer half-life was made.

### Example 22

### Organic Syntheses of 4,5-RCTR1 Analogs

The following synthetic schemes exemplify methods to prepare the 4,5-RCTR1 analogs of interest. Isolation methods include, column chromatography, HPLC, GC, crystallization and distillation if necessary. Characterization can be accomplished by UV, MS, MS/MS, GC/MS, and NMR.

The general synthetic schemes provided below depict methods to prepare the various analogs of 4,5-RCTR1. Throughout the syntheses of these compounds, R groups are used to indicate that various protecting groups can be attached to the hydroxyl group of the 4,5-RCTR1 backbone. This is not to be considered limiting; this is an exemplary protecting group that can be used and was chosen as illustrative.

The moiety designated as “X” is used throughout the synthetic schemes. “X” is meant to include a methyl, ethyl, isopropyl, or trifluoromethyl ester groups, on the carboxylic acid group of the 4,5-RCTR1 carbon chain.

The moiety designated as “Y” is meant to include a methyl, ethyl, isopropyl, or trifluoromethyl ester groups on the 7-glutamyl moiety of the peptido conjugate.

The moiety designated as “Q” is meant to include a methyl, ethyl, isopropyl, or trifluoromethyl ester groups on the cysteinylglycinyl moiety of the peptido conjugate.

Acetylenic portions of the 4,5-RCTR1 early intermediates can provide more additional analogs.

Retention of the triple bonds within the 4,5-RCTR1 backbone is considered advantageous because it shortens the synthesis by eliminating hydrogenation steps.

Scheme II provides a retrosynthetic analysis for the general preparation of 4,5-RCTR1 analogs, from stereochemically pure 4S,5S-epoxy-17S-hydroxy-resolvin methyl ester produced by total organic synthesis. The key step involves the epoxide opening by an amine nucleophile via an S_{N}2 mechanism, in the presence of a Zn^{II} catalyst.

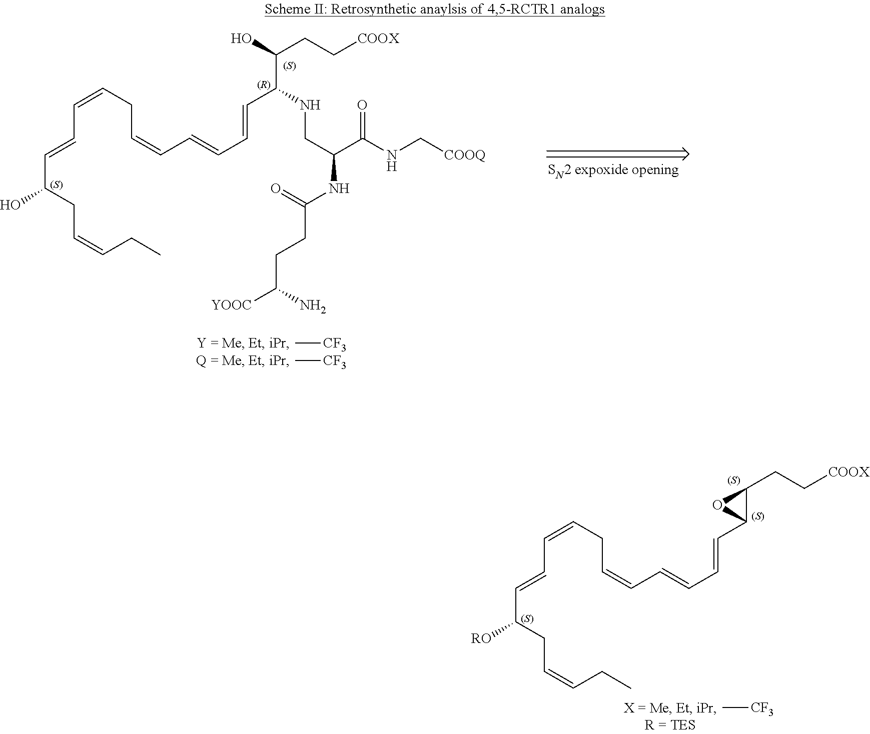

In synthetic Scheme III, the synthesis of 5-amino-substituted-4,5-RCTR1 is achieved via epoxide opening by a modified version of glutathione (GSH). The secondary amine on the γ-glutamyl portion can be protected (e.g., by a carboxybenzyl group, Cbz) to enhance selectivity during epoxide opening. Desilylation with a fluoride followed by hydrolysis under basic conditions cleaves the silyl group and methyl ester, respectively. The Cbz group can be removed via catalytic hydrogenation using palladium on carbon (Pd/C) under mild conditions at neutral pH, leaving acid- or base-sensitive functional groups intact.

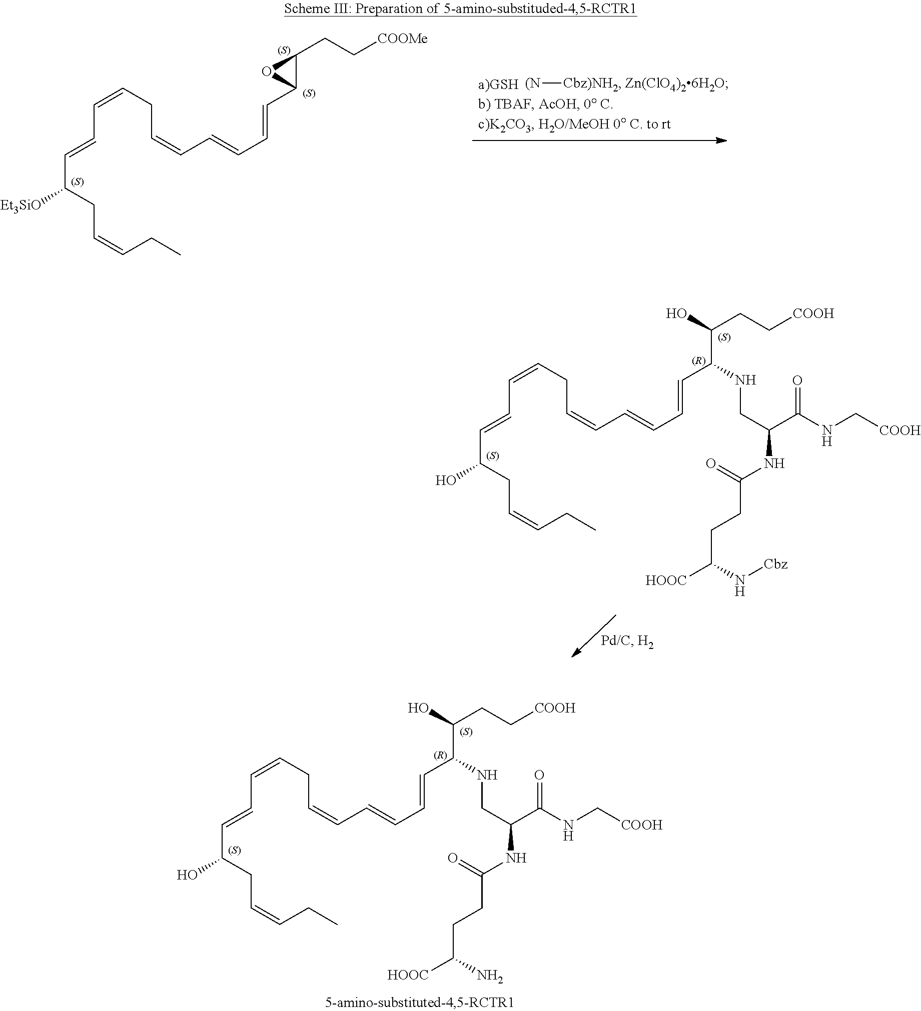

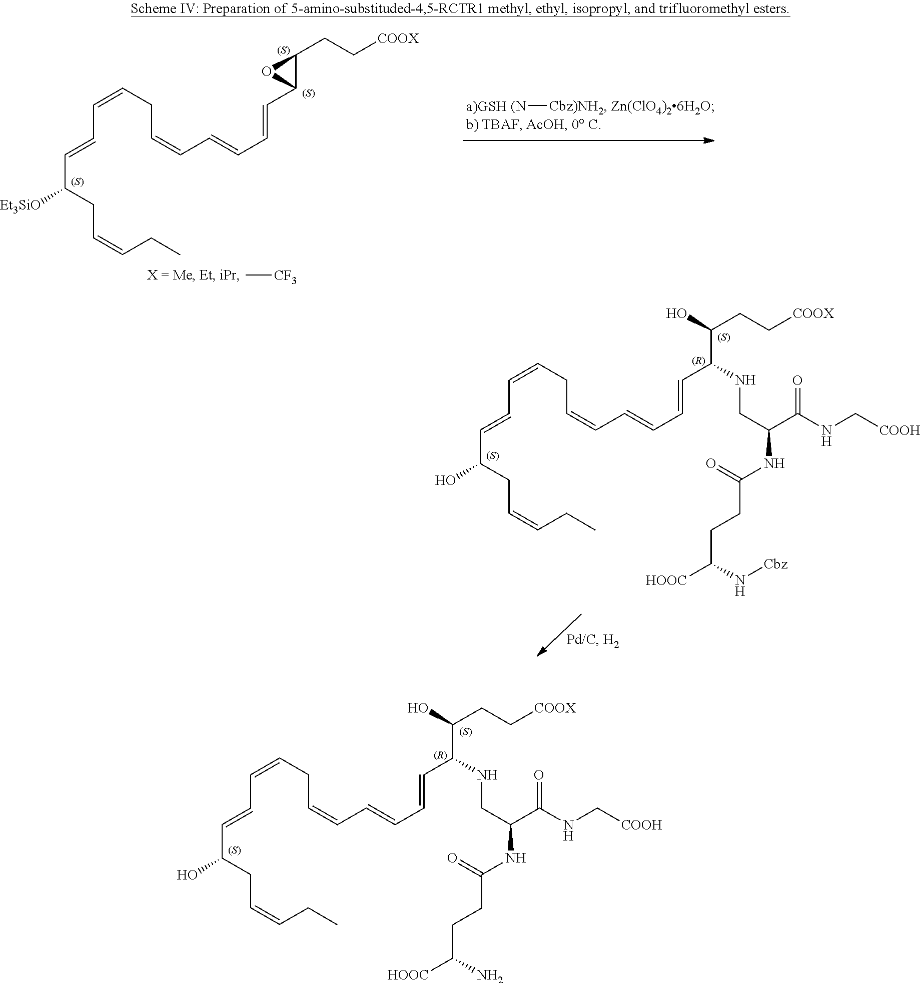

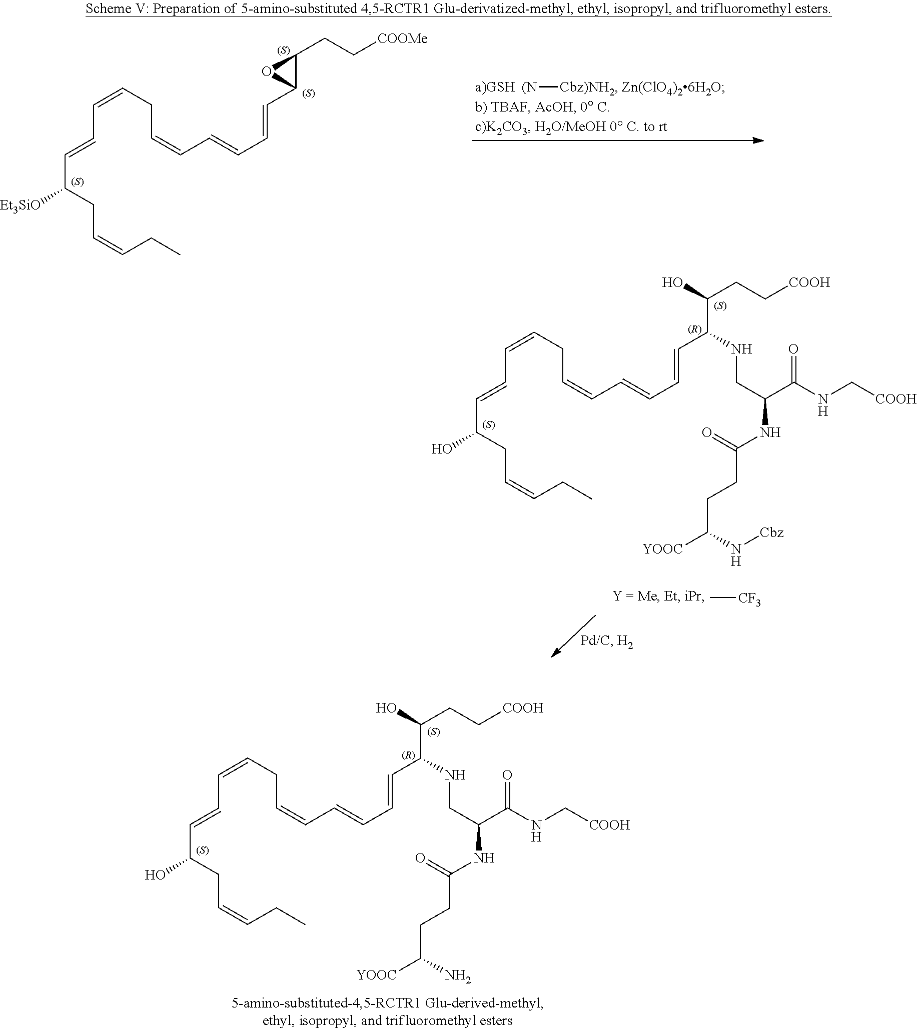

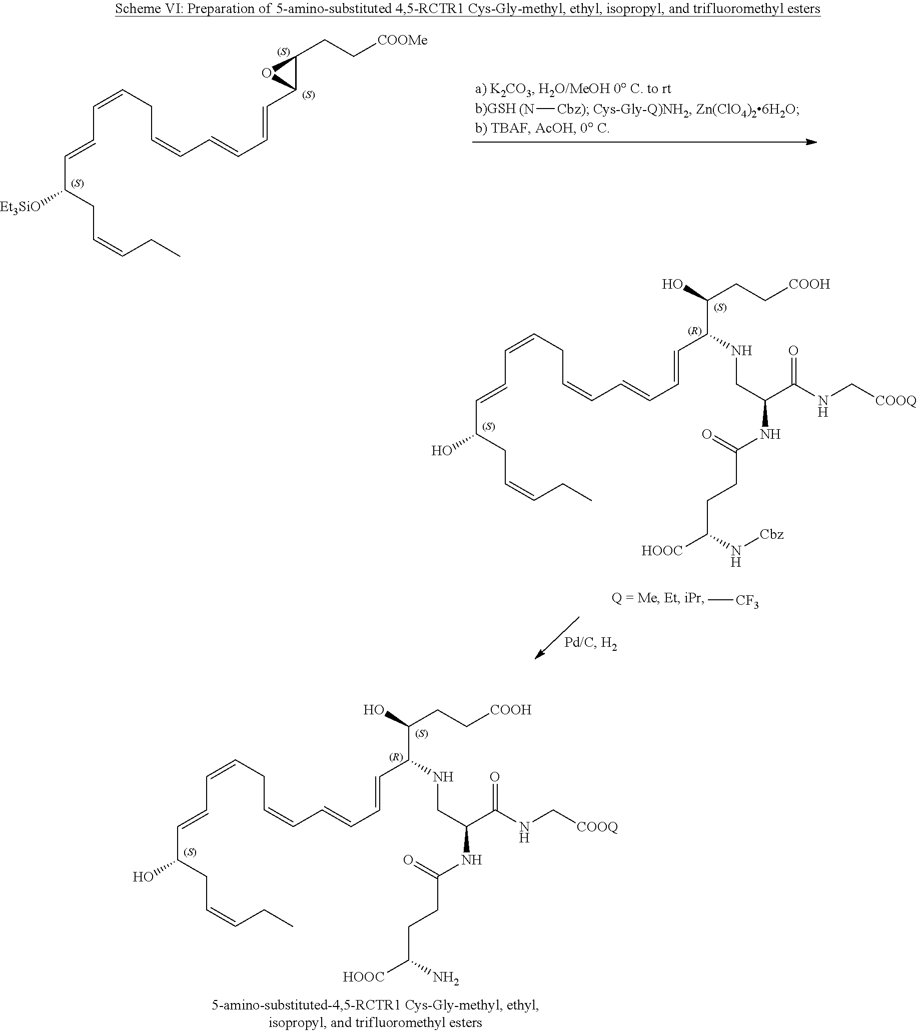

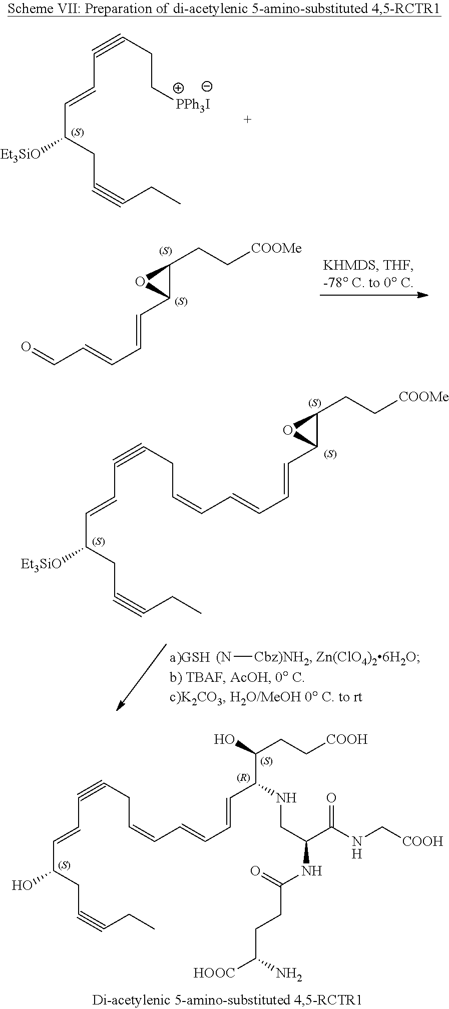

Schemes IV-VII depict carboxylic acid derivatives (methyl, ethyl, isopropyl, and trifluoromethyl esters) at different positions. Namely at the C-1 position of the docosahexaenoate carbon backbone, as well as at the γ-glutamyl and cysteinylglycinyl positions of the peptide moiety. These derivatives are designated herein as “X”, “Y”, and “Q”, respectively, as mentioned above.

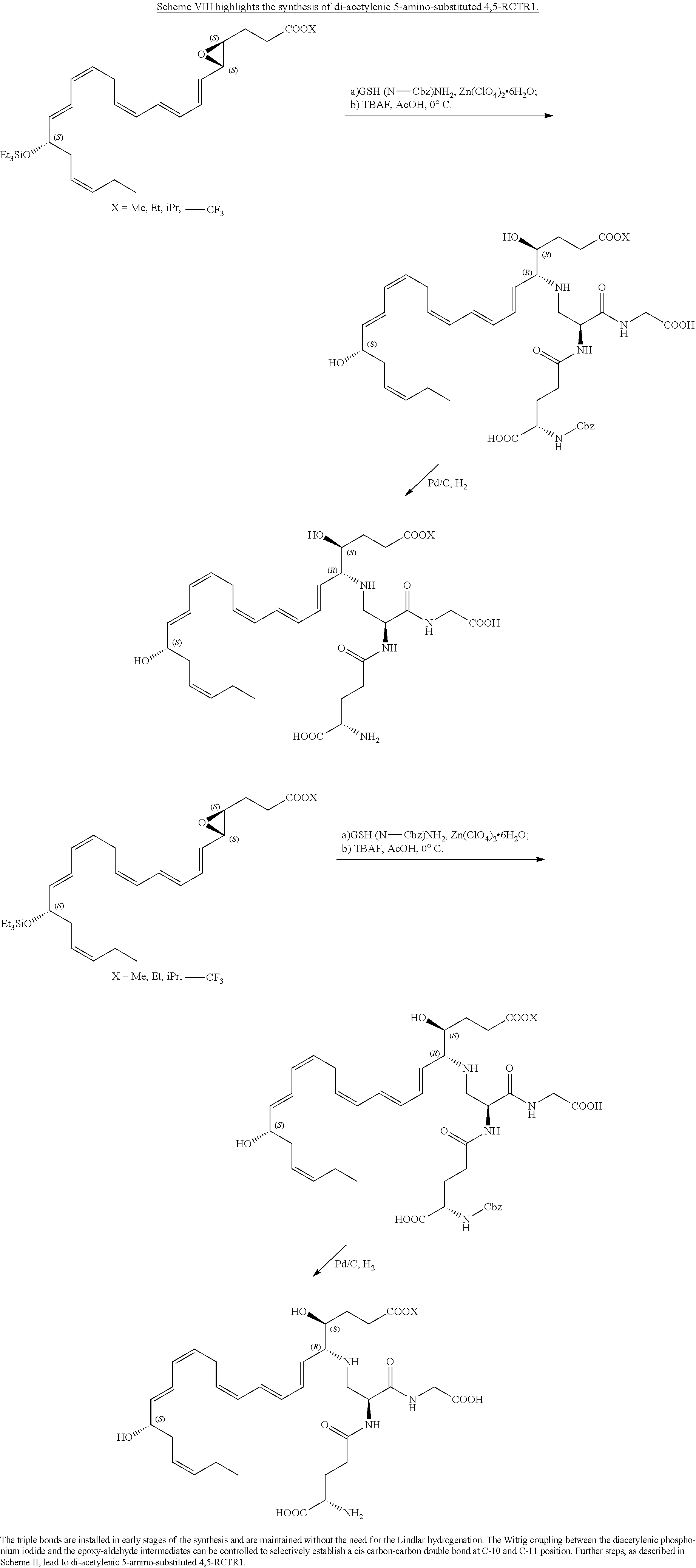

All publications and patents specifically mentioned herein are incorporated by reference for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

The following paragraphs enumerated consecutively from 1-15 provide for various additional aspects of the present invention, in one embodiment, in a first paragraph:

1. A purified compound comprising:
a specialized proresolving mediator (SPM) or SPM derivative conjugated at carbon 4 by an auxochrome to an amino acid, peptide or peptide derivative or a pharmaceutically acceptable salt of the conjugated compound.

2. The purified compound of paragraph 1, wherein the SPM is derived from docosahexaenoic acid.

3. The purified compound of any of paragraphs 1 through 2, wherein the auxochrome is: S, NH, CH₂, or 0.

4. The purified compound of any of paragraphs 1 through 3, wherein the amino acid or peptide derivative is: glutathione.

5. The purified compound of any of paragraphs 1 through 4, having the general formula I, la, II and Ha:

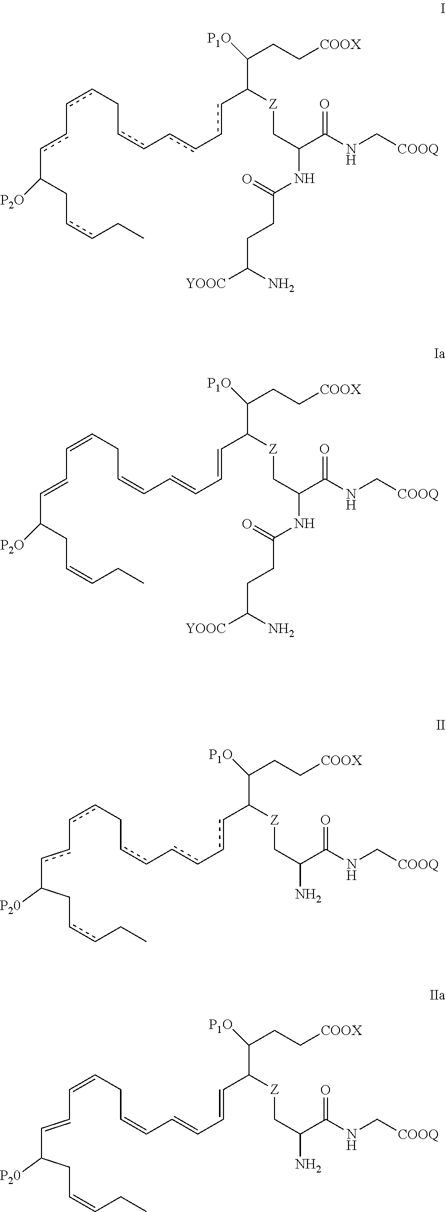

wherein P₁ and P₂ individually are a protecting group or a hydrogen atom;
wherein [symbol] is a double bond;
wherein each double bond is independently in the E or Z configuration;
wherein each Q is independently H, Me, Et, iPr, or —CF3;
wherein each X is independently H, Me, Et, iPr, —CF3,
wherein each Y, when present, is independently H, Me, Et, iPr, —CF3; and
wherein Z is independently S, NH, CH₂, or 0;
optionally wherein when Q, X, and Y are H then P₁ and P₂ cannot both be H if Z is S;
or a pharmaceutically acceptable salt or ester thereof.

6. The compounds of any of paragraphs 1 through 5, wherein the compounds comprise:

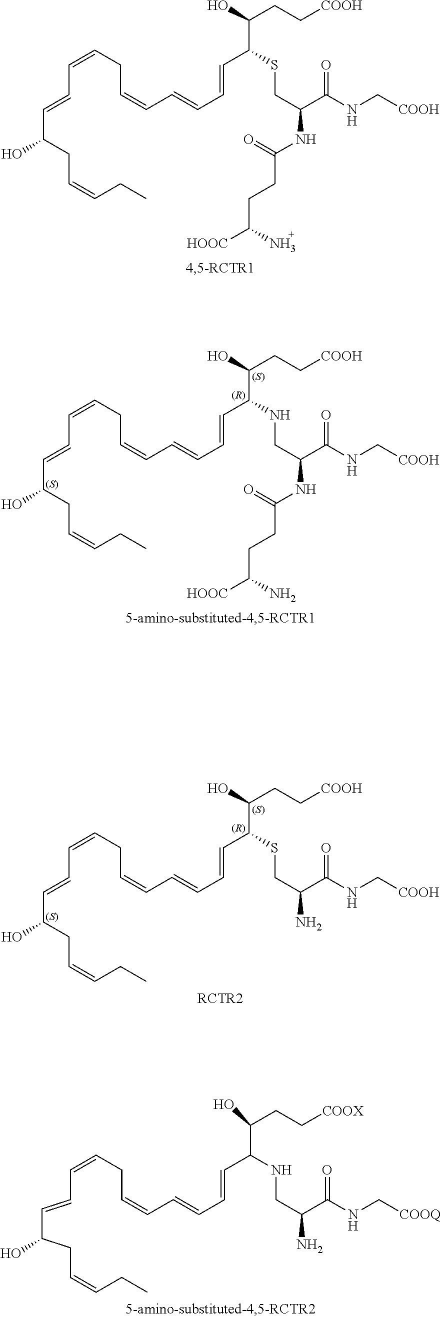

or a pharmaceutically acceptable salt or ester thereof.

7. The compounds of any of paragraphs 1 through 3 wherein the compounds are purified.

8. A composition comprising the compound of any of paragraphs 1 through 3 and a pharmaceutically acceptable carrier.

9. A method of treating inflammation or an inflammatory disease comprising administering to a subject in need thereof a compound or composition according to any of paragraphs 1 through 7.

10. A method for enhancing tissue repair or tissue regeneration comprising, administering to a subject in need thereof a compound or composition according to any of paragraphs 1 through 7.

11. The method of claim 10, wherein enhancing tissue repair or tissue regeneration comprises preventing or ameliorating second organ reperfusion injury.

12. A method of delaying, preventing or treating diseases of granuloma formation comprising, administering to a subject in need thereof a compound or composition according to any of paragraphs 1 through 7.

13. The method of paragraph 12, wherein diseases of granuloma formation comprise: Mycobacterium tuberculosis, chronic granulomatous disease, granulomatous steatitis, foreign-body granulomas, interstitial lung fibrosis (ipf), leprosy, arthritis, sarcoidosis, liver fibrosis, heart fibrosis, renal fibrosis, hepatic cirrhosis, pulmonary fibrosis and organ fibrosis.

14. A method of enhancing resolution of inflammatory diseases wherein the disease comprises: preventing or treating granuloma formation in a subject in need there of comprising administering a compound or composition of any one of paragraphs 1 through 7.

15. The method of paragraph 14, wherein the inflammatory disease comprises: oral inflammation, periodontitis, ulcerative colitis, Cohn's disease, arthritis, asthma and chronic obstructive pulmonary disease, hepatitis, sinusitis, systemic lupus, allergies, dermatitis, atherosclerosis, psoriasis, bronchitis, appendicitis, neurodegenerative diseases, and multiple sclerosis.

What is claimed is:

1. A purified compound comprising:
a specialized proresolving mediator (SPM) or SPM derivative conjugated at carbon 4 by an auxochrome to an amino acid, peptide or peptide derivative or a pharmaceutically acceptable salt of the conjugated compound.

2. The purified compound of claim 1, wherein the SPM is derived from docosahexaenoic acid.

3. The purified compound of claim 2, wherein the auxochrome is: S, NH, CH₂, or O.

4. The purified compound of claim 3, wherein the amino acid or peptide derivative is: glutathione.

5. The purified compound of claim 4, having the general formula I, Ia, II or IIa: 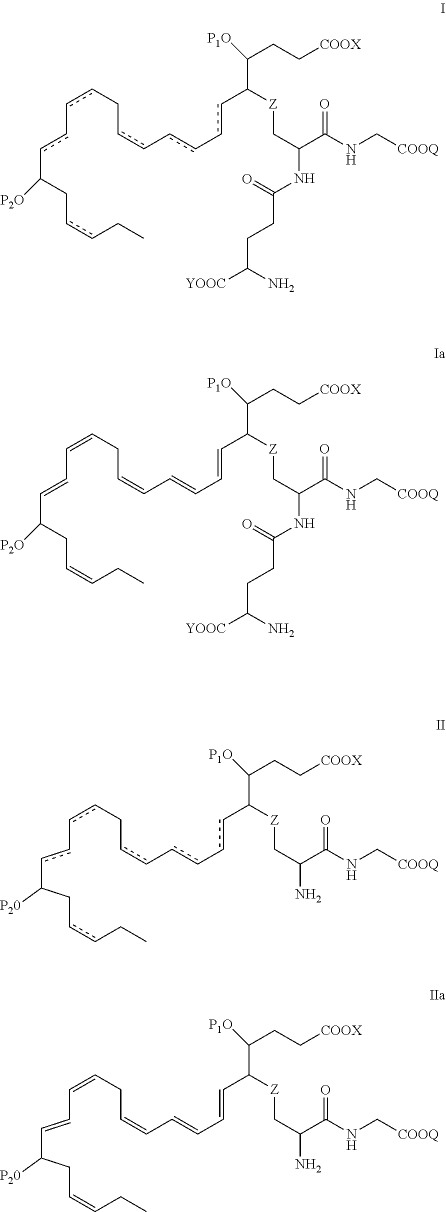
wherein P₁ and P₂ individually are a protecting group or a hydrogen atom;
wherein [symbol] is a double bond;
wherein each double bond is independently in the E or Z configuration;
wherein each Q is independently H, Me, Et, iPr, or —CF3;
wherein each X is independently H, Me, Et, iPr, —CF3,
wherein each Y, when present is independently H, Me, Et, iPr, —CF3; and
wherein Z is independently S, NH, CH₂, or 0;
optionally wherein when Q, X, and Y are H then P₁ and P₂ cannot both be H if Z is S;
or a pharmaceutically acceptable salt or ester thereof.

6. The compound of claim 5, wherein the compound comprises: 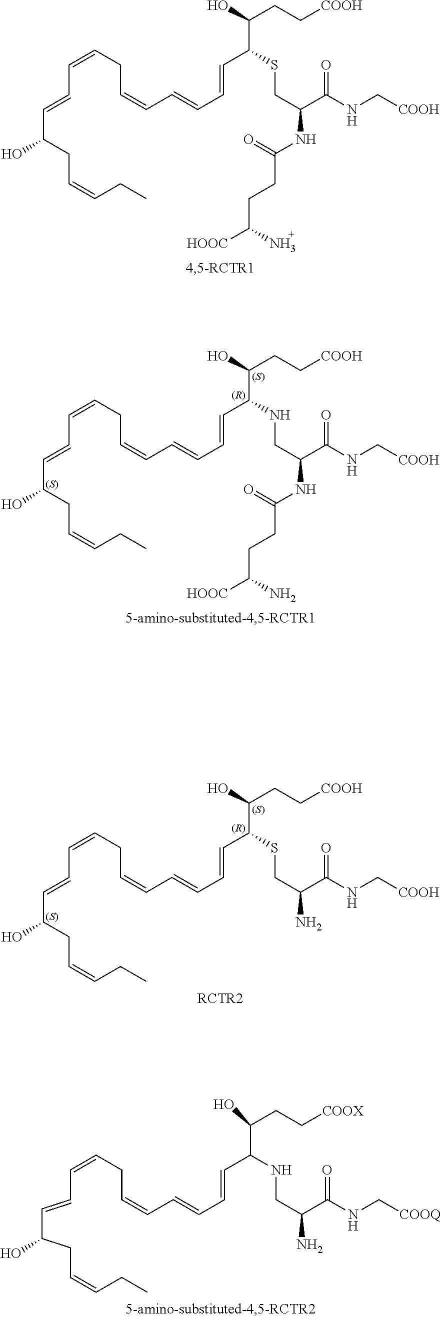
or a pharmaceutically acceptable salt or ester thereof.

7. The compounds of claim 5 wherein the compounds are purified.

8. A composition comprising a compound of claim 1 and a pharmaceutically acceptable carrier.

9. A method of treating inflammation or an inflammatory disease comprising administering to a subject in need thereof a compound or composition according to claim 1.

10. A method for enhancing tissue repair or tissue regeneration comprising, administering to a subject in need thereof a compound or composition according to claim 1.

11. The method of claim 10, wherein enhancing tissue repair or tissue regeneration comprises preventing or ameliorating second organ reperfusion injury.

12. A method of delaying, preventing or treating diseases of granuloma formation comprising, administering to a subject in need thereof a compound or composition according to claim 1.

13. The method of claim 12, wherein diseases of granuloma formation comprise:
Mycobacterium tuberculosis, chronic granulomatous disease, granulomatous steatitis, foreign-body granulomas, interstitial lung fibrosis (ipf), leprosy, arthritis, sarcoidosis, liver fibrosis, heart fibrosis, renal fibrosis, hepatic cirrhosis, pulmonary fibrosis and organ fibrosis.

14. A method of enhancing resolution of inflammatory diseases wherein the disease comprises: preventing or treating granuloma formation in a subject in need there of comprising administering a compound or composition of claim 1.

15. The method of claim 14, wherein the inflammatory disease comprises: oral inflammation, periodontitis, ulcerative colitis, Cohn's disease, arthritis, asthma and chronic obstructive pulmonary disease, hepatitis, sinusitis, systemic lupus, allergies, dermatitis, atherosclerosis, psoriasis, bronchitis, appendicitis, neurodegenerative diseases, and multiple sclerosis.
